(19) United States
(12) Patent Application Publication
(10) Pub. No.: US 2009/0170899 A1
(43) Pub. Date: Jul. 2, 2009

(54) Stimulators of 5-HT4 receptors and uses thereof

(76) Inventors: Guy Debonnel, Mount-Royal Town (CA); Dominique Debonnel, Mount-Royal Town (CA); Guillaume Lucas, Montreal (CA)

Correspondence Address: SMART & BIGGAR;P.O. BOX 2999, STATION D, 900-55 METCALFE STREET, OTTAWA, ON, K1P 5Y6, CA

(21) Appl. No.: 11/662,648

(22) PCT Filed: Sep. 14, 2005
(86) PCT No.: PCT/CA2005/001401

§ 371 Date: Jun. 20, 2008

Related U.S. Application Data
(60) Provisional application No. 60/609,275, filed on Sep. 14, 2004.

(51) Int. Cl. A61K 31/4525; A61K 31/445; A61P 25/24

(52) U.S. Cl. 514/321; 514/320; 514/331

(57) ABSTRACT

The present invention relates to a novel combination of a serotonin selective re-uptake inhibitor (SSRI) and an agonist of the serotonin 4 (5-HT₄) receptor to augment and/or provide faster onset of the therapeutic effect of the SSRI alone or administered with any other compound which causes an elevation in the level of extracellular serotonin (5-HT). The present invention also relates to a pharmaceutical formulation comprising said combination and to a method and use of said combination in the treatment of depression, anxiety, obsessive compulsive disorder (OCD) or other disease or disorder responsive to a SSRI.

### FIELD OF INVENTION

The present invention relates to a novel composition comprising the combination of a selective serotonin reuptake inhibitor (SSRI) and an agonist of the serotonin 4 (5-HT₄) receptor for the use in the treatment of depression, anxiety, obsessive compulsive disorder (OCD) or other disease or disorder responsive to a SSRI.

### BACKGROUND OF INVENTION

It has long been established that serotonergic (5-HT) neurons originating from midbrain raphé nuclei play a major role in numerous functions and are involved in various mental disorders including major depression, anxiety and obsessive-compulsive disorders (Jacobs and Formal, 1999; Blier and de Montigny, 1999). More specifically, regarding depression, several studies have shown that the ability to increase the efficiency of central 5-HT transmission, and particularly in the hippocampal region, constitutes both a common feature of antidepressants (ADs) and a major basis of their therapeutic efficacy (Haddjeri et al., 1998; Blier and de Montigny, 1999). So far, all antidepressant (AD) treatments increase the efficacy of 5-HT transmission at postsynaptic levels (Blier and de Montigny, 1999; Fabre and Hamon, 2003 and Haddjeri et al., 1998). However, nearly all the drugs currently available act by enhancing 5-HT extracellular levels independently from 5-HT neuronal firing rate. This is obviously the case of selective serotonin reuptake inhibitors (SSRIs), the most commonly used antidepressants. Unfortunately, the initial robust elevation of 5-HT concentration triggered by such agents induces the stimulation of 5-HT_{1A} autoreceptors within the dorsal raphé nucleus (DRN) (Mongeau et al., 1997; Blier and de Montigny, 1999). As 5-HT_{3A} autoreceptors exert a strong inhibitory action on 5-HT neuronal activity, their influence counteracts the facilitation of 5-HT transmission related to terminal reuptake blockade (Blier and de Montigny, 1999). The existence of such a presynaptic effect is believed to be responsible for the delay of the therapeutic action of antidepressants, which usually ranges from 3 to 6 weeks (Mongeau et al., 1997; Blier and de Montigny, 1999; Fabre and Hamon, 2003). Indeed, this period corresponds to the time required for 5-HT_{1A} autoreceptors to desensitize (Blier and de Montigny, 1999; Haddjeri et al., 1998). The reduction of this delayed onset of action is still one of the most important challenges in current neuropharmacological research (Blier and de Montigny, 1999). On this basis, it has been proposed that a facilitation of 5-HT activity, independent from desensitization of 5-HT_{1A} autoreceptors, is a requirement for a faster onset of antidepressant action (Blier, 2001). The inventors have recently demonstrated that the activation of 5-HT₄ receptors, through the use of partial or full 5-HT₄ agonists, induces such a facilitation (Lucas and Debonnel, 2002).

In keeping with these observations, a number of typical changes occur in the central 5-HT transmission of AD-administered rodents, within the same time-frame of the delayed therapeutic effect of antidepressants. These modifications include the desensitization of presynaptic 5-HT_{1A} autoreceptors (Blier and de Montigny, 1994, 1999; Mongeau et al., 1997; Blier, 2001; Artigas et al., 2002), and a strong increase of the inhibitory tonus mediated by postsynaptic 5-HT_{1A} receptors, particularly within the hippocampus (Haddjeri et al., 1998; Besson et al., 2000; Blier and Ward, 2003). Long-term treatments with ADs also activate neurogenesis in the hippocampus (Malberg et al., 2000; Nakagawa et al., 2002a; Santarelli et al., 2003). This requires the presence of 5-HT_{1A} receptors, and recent work indicates that activation of adult hippocampal neurogenesis may help mediate the beneficial action of ADs (Santarelli et al., 2003; Castrén, 2004). Several studies have suggested that AD-induced neurogenesis is related to an elevation of the cAMP response element-binding protein (CREB) concentration in hippocampal neurons, and more specifically, on its phosphorylation into PCREB (Nibuya et al., 1996; Thome et al., 2000; Nakagawa et al., 2002a; Tiraboschi et al., 2004).

Accordingly, there is a need for a 5-HT₄ agonist or a pharmaceutically acceptable salt thereof useful for augmenting and/or providing faster onset of the therapeutic effect of a SSRI alone or administered with any other compound that causes an elevation in the level of extracellular serotonin agents. More specifically, there is a need for a pharmaceutical combination comprising a 5-HT₄ agonist and a SSRI which has broad clinical usefulness for the treatment of depression and other affective disorders. As a consequence, combination therapy using a 5-HT₄ agonist and a SSRI may be effective in reducing or preventing altogether the side-effects associated with the SSRI when used in monotherapy.

### SUMMARY OF INVENTION

The present invention is directed, in part, to a combination of a selective serotonin reuptake inhibitor (SSRI) and a 5-HT₄ receptor agonist. More specifically, the present invention relates to the 5-HT₄ agonist, or a pharmaceutically acceptable salt thereof, in combination with a SSRI alone or administered with any other compound that causes an elevation in the level of extracellular serotonin (5-HT), to augment and/or provide faster onset of the therapeutic effect of the SSRI. As used herein, the term “augment” covers improving the therapeutic effect and/or potentiating the therapeutic effect of a SSRI alone or administered with any other compound that causes an elevation in the level of extracellular 5-HT.

In a further embodiment, the invention relates to the use of a 5-HT₄ agonist, or a pharmaceutically acceptable salt thereof and a SSRI alone or administered with any other compound that causes an elevation in the level of extracellular serotonin, for the preparation of a pharmaceutical composition for the treatment of depression, anxiety, obsessive compulsive disorder (OCD) or other disease or disorder responsive to a SSRI.

In one embodiment, the present invention relates to the use of a 5-HT₄ agonist, or a pharmaceutically acceptable salt thereof and a SSRI alone or administered with any other compound that causes an elevation in the level of extracellular serotonin, for the preparation of a pharmaceutical composition that is adapted for simultaneous administration of the active ingredients. In particular, the pharmaceutical compositions may contain the active ingredients within the same unit dosage form, e.g. in the same tablet or capsule. Such unit dosage forms may contain the active ingredients as a homogenous mixture or in separate compartments of the unit dosage form.

In another embodiment, the present invention relates to the use of a 5-HT₄ agonist, or a pharmaceutically acceptable salt thereof and a SSRI alone or administered with any other compound that causes an elevation in the level of extracellular serotonin, for the preparation of a pharmaceutical composition that is adapted for sequential administration of the active ingredients. In particular, the pharmaceutical composition may contain the active ingredients in discrete unit dosage form, e.g. discrete tablets or capsules containing either of the active ingredients. These discrete unit dosage forms may be contained in the same container or package.

In a further embodiment, the present invention relates to the use of a 5-HT₄ agonist, or a pharmaceutically acceptable salt thereof, for augmenting and/or providing faster onset of the therapeutic effect of a SSRI alone or administered with any other compound that causes an elevation in the level extracellular serotonin, to a subject to be treated with, or undergoing treatment with the SSRI. For example, the 5-HT₄ agonist may be used as add-on therapy for the augmentation of the response to a SSRI in an individual or subject where a reduction in symptoms has not been achieved during the first 6 weeks of treatment with a SSRI.

A further embodiment of the present invention is a package containing a combination of a SSRI and a 5-HT₄ agonist, as well as instructions for use. In particular, the combination may contain the SSRI and a 5-HT₄ agonist in the same unit dosage form, e.g. in the same tablet or capsule, for simultaneous administration of the combination. Such unit dosage forms may contain the SSRI and a 5-HT₄ agonist as a homogenous mixture or in separate compartments of the unit dosage form. Alternatively, the package may contain the SSRI and a 5-HT₄ agonist in two discrete unit dosage forms in the same container or package, e.g. discrete tablets or capsules, for sequential administration of the combination.

The present invention also relates to a method for augmenting and/or providing faster onset of the therapeutic effect of a SSRI alone or administered with any other compound that causes an elevation in the level extracellular serotonin, comprising administering a 5-HT₄ agonist, or a pharmaceutically acceptable salt thereof to a subject to be treated with, or undergoing treatment with the SSRI.

The invention also relates to a method of treating depression, anxiety or other affective disorder responsive to a SSRI, or any other compound that causes an elevation in the level of extracellular serotonin, comprising administering to a subject in need thereof: (a) a therapeutically effective amount of a SSRI alone or administered with any other compound, that causes an elevation in the level of extracellular serotonin, to treat depression, anxiety, obsessive compulsive disorder (OCD) or other disease or disorder responsive to a SSRI; and (b) a therapeutically effective amount of a 5-HT₄ agonist, or pharmaceutically acceptable salt thereof, to augment and/or provide faster onset of the therapeutic effect of the SSRI, or any other compound, that causes an elevation in the level of extracellular serotonin.

A “therapeutically effective amount” refers to an amount effective, at dosages and for periods of time necessary, to achieve the desired therapeutic result, such as to augment and/or provide faster onset of the therapeutic effect of the SSRI. A therapeutically effective amount of a 5-HT₄ agonist and a SSRI may vary according to factors such as the disease state, age, sex, and weight of the individual, and the ability of the compound to elicit a desired response in the individual. Dosage regimens may be adjusted to provide the optimum therapeutic response. A therapeutically effective amount is also one in which any toxic or detrimental effects of the compound are outweighed by the therapeutically beneficial effects.

According to a first preferred embodiment, the present invention relates to prucalopride, or a pharmaceutically acceptable salt thereof in combination with a SSRI, preferably citalopram, alone or administered with any other compound that causes an elevation in the level of extracellular serotonin, for augmenting and/or providing faster onset of the therapeutic effect of the SSRI.

According to a second preferred embodiment, the present invention relates to RS 67333, or a pharmaceutically acceptable salt thereof in combination with a SSRI, preferably paroxetine, alone or administered with any other compound that causes an elevation in the level of extracellular serotonin, for augmenting and/or providing faster onset of the therapeutic effect of the SSRI.

The term “selective serotonin reuptake inhibitor” or “SSRI” will both be used hereunder. The terms refer to compounds that selectively inhibit the reuptake of serotonin. This family of compounds generally presents only a low affinity for most of the subtype of serotonin receptors, but block selectively the selective reuptake sites for serotonin. Through this effect, they therefore increase synaptic availability of 5-HT for postsynaptic receptors. This increase in synaptic 5-HT is measurable and has been used as an index of the efficacy of SSRIs. The 5-HT transporter protein have sufficient affinity for SSRIs to enable binding studies which can be carried out with tritiated ligands such as [³H]-imipramine, [³H]-paroxetine or [³H]-citalopram. These radioligand binding studies constitute a second way to assess the efficacy of SSRIs. These, and other tests to determine whether a compound is a selective serotonin reuptake inhibitor are well known in the art (see, for example, Ross, S. B. and A. L. Ask. (1980) Structural requirements for uptake into serotoninergic neurones. Acta Pharmacol. Toxicol. (Copenh) 46.4:270-277).

The term “5-HT₄ receptor agonist” or “5-HT₄ agonist” as used herein refers to a compound, which activates a 5-HT₄ receptor under complete or partial activation.

The individual or subject which may benefit from treatment with a combination as above, may suffer from depression, anxiety, obsessive compulsive disorder (OCD) or other disease or disorder responsive to SSRI therapy. The terms “individual” or “subject” refers to warm blooded animals such as, for example, guinea pigs, mice, rats, cats, rabbits, dogs, monkeys, chimpanzees, and humans

As used in this application, the term “depression” should be construed as encompassing those conditions which the medical profession have referred to as major depression, endogenous depression, psychotic depression, involutional depression, involutional melancholia, disthymia, etc. These conditions are used to describe a condition in which patients typically experience intense sadness and despair, mental slowing, loss of concentration, pessimistic worry, despair, and agitation. The patients often experience physical complaints such as insomnia, anorexia, decreased energy, decreased libido, etc.

The term “anxiety” refers to the unpleasant emotional state consisting of psychophysiological responses to anticipation of unreal or imagined danger, ostensibly resulting from unrecognized intrapsychic conflict. Physiological concomitants include increased heart rate, altered respiration rate, sweating, trembling, weakness, and fatigue; psychological concomitants include feelings of impending danger, powerlessness, apprehension, and tension.

In connection with the use of the 5-HT₄ agonist with a SSRI, for the treatment of subjects possessing any of the above conditions, it is to be noted that these agents may be administered either alone or in combination with pharmaceutically acceptable carriers suitable for administration orally, rectally, or by parenteral injection, and that such administration can be carried out in both single and multiple dosages. More particularly, the 5-HT₄ agonist and SSRI can be administered in a wide variety of different dosage forms, i.e., they may be combined with various pharmaceutically-acceptable inert carriers in the form of tablets, capsules, lozenges, troches, hand candies, powders, sprays, aqueous suspension, injectable solutions, elixirs, syrups, and the like. Such carriers include solid diluents or fillers, sterile aqueous media and various non-toxic organic solvents, etc. Suitable pharmaceutical carriers are described in “Remington's Pharmaceutical Sciences” by E. W. Martin, 18th Edition.

The phrase “pharmaceutically acceptable”, as used in connection with compositions of the invention, refers to molecular entities and other ingredients of such compositions that are physiologically tolerable and do not typically produce untoward reactions when administered to a subject or individual.

The term “combination” applied to active ingredients is used herein to define a single pharmaceutical composition (formulation) comprising both drugs of the invention (i.e. 5-HT₄ agonist and a SSRI) or two separate pharmaceutical compositions (formulations), each comprising a single drug of the invention (i.e. 5-HT₄ agonist and a SSRI), to be administered conjointly.

Within the meaning of the present invention, to be administered “conjointly” refers to administration of the 5-HT₄ agonist and a SSRI simultaneously in one composition, or simultaneously in different compositions, or sequentially. For the sequential administration to be considered “conjoint”, however, the 5-HT₄ agonist and a SSRI must be administered separated by a time interval that still permits the resultant beneficial effect for treating, preventing, arresting a symptom or behavior associated with depression, anxiety, obsessive compulsive disorder (OCD) or other disease or disorder responsive to a SSRI in a subject or individual and provide a faster onset of action of the SSRI.

It will also be appreciated that when using a combination method of the present invention, referred to above, both the 5-HT₄ agonist and the SSRI will be administered to a subject within a reasonable period of time. The compounds may be in the same pharmaceutically acceptable carrier and therefore administered simultaneously. They may be in separate pharmaceutical carriers such as conventional oral dosage forms that are taken simultaneously. The term combination, as used above, also refers to the case where the compounds are provided in separate dosage forms and are administered sequentially. Therefore, by way of example, the SSRI may be administered as a tablet and then, within a reasonable period of time, the 5-HT₄ receptor agonist may also be administered as a tablet.

Other objects, features, advantages and aspects of the present invention will become apparent to those of skill from the following description. It should be understood, however, that the following description and the specific examples, while indicating preferred embodiments of the invention, are given by way of illustration only. Various changes and modifications within the spirit and scope of the disclosed invention will become readily apparent to those skilled in the art from reading the following description and from reading the other parts of the present disclosure.

### BRIEF DESCRIPTION OF THE DRAWINGS

The invention is illustrated below with reference to the following figures:

FIGS. 1A and 1B illustrate the effects of subacute and chronic administrations of both prucalopride (2.5 mg/kg) and RS 67333 (1.5 mg/kg) on the increase of DRN 5-HT activity;

FIGS. 2A to 2D illustrate the effects of an acute, i.v. administration of GR 125487 on the increase of DRN 5-HT neuron firing rate induced by 3-day treatments with either prucalopride or RS 67333;

FIGS. 3A and 3B show the effects of citalopram and prucalopride, alone or in combination, on the total time of immobility and climbing behavior evaluated in the forced swimming test (FST);

FIGS. 4A and 4B show the effects of citalopram, prucalopride, and RS 67333 separately on the total time of immobility and climbing behavior evaluated in the forced swimming test (FST);

FIGS. 5A and 5B show the effects of paroxetine and RS 67333, alone or in combination, on the total time of immobility and climbing behavior evaluated in the forced swimming test (FST); and

FIGS. 6A and 6B illustrate the effect of prucalopride on the inhibition of 5-HT neuronal firing rate induced by SSRIs fluxoamine and citalopram.

FIG. 7 is a dose-response curve illustrating the effect of cumulative intravenous doses of citalopram on DRN 5-HT neuron average (mean±S.E.M.) firing rate, expressed as percentage of basal activity, in rats treated with the selective 5-HT₄ agonist RS 67333 (1.5 mg/kg/day, 3 days, n=5) or its vehicle (n=5). Single-cell extracellular recordings were performed in the DRN of chloral-hydrate anaesthetized rats by using single-barrel glass microelectrodes, and 5-HT neurons were identified according to the classical criteria (see Example 5, the experimental procedures). RS 67333 was administered through the use of osmotic minipumps, inserted subcutaneously in the region of the back. Recordings were performed with the minipumps still in place.

FIGS. 8A to 8C. FIG. 8A illustrates the effect of cumulative intravenous doses of the selective 5-HT_{1A} antagonist WAY 100635 on the average (mean±S.E.M.) firing activity of hippocampal pyramidal neurons of the CA3 sub-field, in rats treated with the selective 5-HT₄ agonists RS 67333 (1.5 mg/kg/day, 3 days, n=5) and prucalopride (2.5 mg/kg/day, 3 days, n=5), or their vehicle (n=5). Single-cell extracellular recordings were performed in the CA₃ region of chloral-hydrate anaesthetized rats by using multiple-barrel glass microelectrodes, combined with microiontophoretic pumps (with the central barrel used for recordings). Pyramidal neurons were identified according to the classical criteria (see Example 5, the experimental procedures). The 5-HT₄ agonists were administered through the use of osmotic minipumps, inserted subcutaneously in the region of the back. Recordings were performed with the minipumps still in place. *p<0.05, **p<0.01, ***p<0.001 vs control, Dunnett's test. FIG. 8B shows a typical example (integrated firing rate histogram) of the response of a single pyramidal neuron to cumulative doses of WAY 100635, in the RS 67333-treated group. High values of ejection currents were required for quisqualate. FIG. 8C is a typical example showing the response of a single pyramidal neuron to cumulative doses of WAY 100635, in a rat co-administered with RS 67333 (same treatment as above for FIG. 8A) and the 5-HT depleter para-chlorophenylalanine (pCPA; 150 mg/kg, i.p. once daily 72, 48 and 24 h before the recordings). In this case, the ejection currents required for quisqualate were similar to those used in control animals.

FIGS. 9A to 9D. FIGS. 9A and 9C show a typical example (integrated firing rate histogram) of the response of a single hippocampal pyramidal neuron of the CA₃ sub-field to an acute, intravenous administration of the selective 5-HT₄ agonist prucalopride (1000 μg/kg), and its reversal by local, microiontophoretic application of the selective 5-HT_{1A} antagonist WAY 100635 (A), or systemic injection of the selective 5-HT₄ antagonist GR 125487 (1000 μg/kg, i.v.) (C). Single-cell extracellular recordings were performed in the CA₃ region of chloral-hydrate anaesthetized rats by using multiple-barrel glass microelectrodes, combined with microiontophoretic pumps (with the central barrel used for recordings). Pyramidal neurons were identified according to the classical criteria (see Example 5, experimental procedures). FIGS. 9B and 9D show a summary of the effects as for FIGS. 9A and 9C, respectively. Bar histograms represent the mean (S.E.M.) percentage effect, calculated for each neuron with respect to its basal firing rate (i.e. 100% value), and the number in each column represents the number of neurons tested. *p<0.05 vs basal value, one-way ANOVA.

FIG. 10 illustrates the effect of the selective 5-HT₄ agonists RS 67333 (1.5 mg/kg/day, 3 days) and prucalopride (2.5 mg/kg/day, 3 days) on the activation of CREB in hippocampal tissue, assessed by measuring phosphoCREB (PCREB) immunoreactivity. CREB phosphorylation was normalized according to the amount of protein present in each sample by expressing the data as a ratio of pCREB over total CREB immunoreactivity (see Example 5, the experimental procedures for more details). Results represent mean±SEM of at least seven experiments. Inset shows representative examples of PCREB immunoreactivity for different treatment conditions indicated on the histogram. The 5-HT₄ agonists were administered through the use of osmotic minipumps, inserted subcutaneously in the region of the back. *p<0.05 vs vehicle, one-way ANOVA.

FIG. 11 shows the effect of RS 67333 (1.5 mg/kg/day, 3 days) on the number of BrdU-positive cells in the sub-granular zone (SGZ) of the hippocampus. A and B: Photomicrographs (magnification: 10× and 100× for the small rectangles) representative of the control and RS 67333 group, respectively. Clusters of positive cells were found in the presence of RS 67333 (small rectangles). RS 67333 was administered through the use of osmotic minipumps, inserted subcutaneously in the region of the back. Rats received intraperitoneal injections of BrdU at the dose of 50 mg/kg twice daily (8 h interval), starting from the day of minipump insertion until day 2 post-surgery. Inset shows the summary (mean±S.E.M.) of the effect of RS 67333, expressed as density of BrdU-positive cells per mm² (n=4 in each group). **p<0.01 vs control, one-way ANOVA.

FIGS. 12A to 12C. FIG. 12A shows the effect of the selective 5-HT₄ agonists RS 67333 (1.5 mg/kg, i.p.) and prucalopride (2.5 mg/kg, i.p.) on the time spent immobile in the FST. All data are expressed as mean±S.E.M. of eight animals per group, and are from an observation of 4 min duration. Rats experienced a pre-test session (15 min) 24 h before the test session. The 5-HT₄ agonists were administered 30 min before the test session. FIG. 12B shows the effect of RS 67333 and prucalopride on the time spent climbing in the FST, in the same animals as used in FIG. 12A. FIG. 12C shows the effect of RS 67333 (1.5 mg/kg, i.p.) and prucalopride (2.5 mg/kg, i.p.) on the horizontal locomotion in activity chambers, equipped with photoelectric cells to allow activity counts. Data are expressed as mean±S.E.M. of eight animals per group, and are from an observation of 10 min duration. The 5-HT₄ agonists were administered 30 min before testing. *p<0.05, **p<0.01 vs the vehicle, Dunnett's test;

FIG. 13 shows the effects of citalopram (10 mg/kg, i.p.) and prucalopride (2.5 mg/kg, i.p.), alone or in combination, on the total time of immobility and climbing behavior evaluated in the forced swimming test (FST);

FIGS. 14A to 14C. FIG. 14A shows the effects of paroxetine (10 mg/kg, i.p.) and RS 67333 (1.5 mg/kg, i.p.), alone or in combination, on the total time of immobility and climbing behavior evaluated in the forced swimming test (FST). FIG. 14B shows the effects of fluvoxamine (10 mg/kg, i.p.) and RS 67333 (1.5 mg/kg, i.p.), alone or in combination, on the total time of immobility and climbing behavior evaluated in the forced swimming test (FST). FIG. 14C shows the effects of fluoxetine (10 mg/kg,i.p.) and RS 67333 (1.5 mg/kg, i.p.), alone or in combination, on the total time of immobility and climbing behavior evaluated in the forced swimming test (FST);

FIGS. 15A to 15B. FIG. 15A shows the effect of citalopram (3 mg/kg, i.p.) and RS 67333 (1.5 mg/kg, i.p.), alone or in combination, on the total time of immobility and climbing behavior evaluated in the forced swimming test (FST). FIG. 15B shows the effect of citalopram (10 mg/kg, i.p.) and RS 67333 (1.5 mg/kg, i.p.), alone or in combination, on the total time of immobility and climbing behavior evaluated in the forced swimming test (FST); and

FIGS. 16A to 16B. FIG. 16A shows the effects of citalopram (10 mg/kg/day) and prucalopride (2.5 mg/kg/day), alone or in combination, on 5-HT_{1A} tonus in the hippocampus in response to WAY 100635. FIG. 16B shows the effects of RS 67333 (1.5 mg/kg/day) under the same conditions.

### DETAILED DESCRIPTION OF THE INVENTION

As discussed above, SSRIs show a delayed onset of action such that several weeks of treatment are necessary to achieve a relief in symptoms. Advantageously, treatment with a SSRI in combination with a 5-HT₄ agonist should result in an increased synaptic concentration of serotonin (5-HT) through the direct facilitation or enhancement of 5-HT neuronal activity. Accordingly, the 5-HT₄ agonists have a clinical potential to improve the efficacy of the SSRI and offer a new approach for rapid onset of affective therapeutic action for the treatment of depression, anxiety or affective disorder which cannot be treated appropriately by administration of a SSRI alone. Typically, a 5-HT₄ agonist may be used as add-on or adjunct therapy for the augmentation of the response to a SSRI in an individual or subject where a reduction in symptoms has not been achieved during the first 6 weeks of treatment with a SSRI alone.

The combination according to the present invention may exist in one pharmaceutical formulation comprising both the SSRI and the 5-HT₄ agonist, or in two different pharmaceutical formulations, one for the SSRI and one for the 5-HT₄ agonist. The pharmaceutical formulation may be in the form of tablets or capsules, powders, mixtures, solutions or other suitable pharmaceutical formulation forms.

It will be understood by the skilled reader that all of the compounds used in the present invention (e.g. the 5-HT₄ agonist and SSRI) are capable of forming salts, and that the salt forms of pharmaceuticals are commonly used, often because they are more readily crystallized and purified than are the free bases. In all cases, the use of the pharmaceuticals described above as salts is contemplated in the description herein, and often is preferred, and the pharmaceutically acceptable salts of all of the compounds are included in the names of them.

The term “pharmaceutically acceptable salts” or “a pharmaceutically acceptable salt thereof” refer to salts prepared from pharmaceutically acceptable nontoxic acids or bases including inorganic acids and bases. Suitable pharmaceutically acceptable acid addition salts for the compounds used in the present invention may include acetic, benzenesulfonic (besylate), benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric acid, p-toluenesulfonic, and the like. In all cases, the use of the pharmaceuticals described above as salts is contemplated in the description herein, and often is preferred, and the pharmaceutically acceptable salts of all of the compounds are included in the names of them.

Many SSRIs have been described in the literature. Any pharmacologically active compound, which primarily or partly exert its therapeutic effect via inhibition of selective serotonin reuptake in the central nervous system (CNS), may benefit from augmentation with a 5-HT₄ agonist. The term “selective serotonin reuptake inhibitors” or “SSRIS” may also refer to prodrugs or forms that may release the actual active ingredient.

Known SSRIs which may benefit from augmentation of a 5-HT₄ agonist are citalopram, escitalopram, fluoxetine, fluvoxamine, sertraline, paroxetine, zimeldine, norzimeldine, clomipramine, alaproclate, venlafaxine, cericlamine, duloxetine, milnacipran, nefazodone, OPC 14503, and cyanodothiepin, preferably citalopram, fluvoxamine, paroxetine, and fluoxetine. However, these SSRIs and other compounds, which cause an increase in the extracellular level of serotonin, are not to be construed as limiting. The definitions and chemical names of the aforementioned SSRIs can be found in the Merck Index, 12^{th} Ed., S. Budovari, et al. (eds) and are incorporated herein by reference.

SSRIs differ both in molecular weight and in activity. As a consequence, the amount of SSRI used in combination therapy depends on the nature of the SSRI.

The term “5-HT₄ receptor agonist” or “5-HT₄ agonist” as used herein refers to a compound which activates a 5-HT₄ receptor under complete or partial activation. Examples of these compounds include, but are not limited to, prucalopride, RS 67333 (1-(4-amino-5-chloro-2-methoxyphenyl)-3-(1-n-burtl-4-piperidinyl)-1-propanone), RS 67506 (1-(4-amino-5-chloro-2-methoxyphenyl)-3-[1-[2-[(methylsulfonyl)amino]ethyl]-4-piperidinyl]-1-propanone), cisapride, renzapride, norcisapride, mosapride, zacopride, tegaserod, SB 205149, SC 53116, BIMU 1 and BIMU 8. In a preferred embodiment, the 5-HT₄ agonist is prucalopride or RS 67333.

The 5-HT₄ agonist may be administered before, during or after the administration of the SSRI provided that the time between the administration of 5-HT₄ agonist and the administration of the SSRI such that these ingredients are allowed to provide a faster onset of the therapeutic effect of a SSRI alone. When simultaneous administration of the 5-HT₄ agonist and a SSRI is envisaged, a composition containing both a SSRI and a 5-HT₄ agonist may be particularly convenient. Alternatively, the 5-HT₄ agonist and the SSRI may be administered separately in the form of suitable compositions. The compositions may be prepared as described below.

To prepare the pharmaceutical compositions of this invention, an effective amount of a particular compound, in base or acid addition salt form, as the active ingredient is combined in intimate admixture with a pharmaceutically acceptable carrier, which carrier may take a wide variety of forms depending on the form of preparation desired for administration. These pharmaceutical compositions are desirably in unitary dosage form suitable, preferably, for administration orally, rectally, or by parenteral injection. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions: or solid carriers such as starches, sugars; kaolin, lubricants, binders, disintegrating agents and the like in the case of powders, pills, capsules and tablets. Because of their ease in administration, tablets and capsules represent the most advantageous oral dosage unit form, in which case solid pharmaceutical carriers are obviously employed. For parenteral compositions, the carrier will usually comprise sterile water, at least in large part, though other ingredients, to aid solubility for example, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed.

It is especially advantageous to formulate the aforementioned pharmaceutical compositions in dosage unit form for ease of administration and uniformity of dosage. As used in the specification and claims, unit dosage form refers to physically discrete units suitable as unitary dosages, each unit containing a predetermined quantity of active ingredient(s) calculated to produce the desired therapeutic effect, in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof.

To produce pharmaceutical formulations of the combination of the invention in the form of dosage units for oral administration, tablets containing various solid excipients such as lactose, saccharose, sorbitol, mannitol, starches such as potato starch, corn starch or amylopectin, cellulose derivatives, a binder such as gelatine or polyvinylpyrrolidone, disintegrants e.g. sodium starch glycolate, cross-linked PVP, cross-caramellose sodium and a lubricant such as magnesium stearate, calcium stearate, polyethylene glycol, waxes, paraffin, and the like, and then compressed into tablets. Additionally, lubricating agents such as magnesium stearate, sodium lauryl sulfate and talc are often very useful for tabletting purposes.

Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the active ingredient may be combined with various sweetening or flavoring agents, coloring matter or dyes, and, if so desired, emulsifying and/or suspending agents as well, together with such diluents as water, ethanol, propylene glycol, glycerin and various like combinations thereof. Usually the active ingredients in the combination will constitute between 0.1 and 99% by weight of the formulation, more specifically between 0.5 and 20% by weight for formulations intended for injection and between 0.2 and 50% by weight for formulations suitable for oral administration.

Dosage units for rectal application can be solutions or suspensions or can be prepared in the form of suppositories comprising the active substances in a mixture with a neutral fatty base, or gelatin rectal capsules comprising the active substances in admixture with vegetable oil or paraffin oil. Liquid formulations for oral application may be in the form of syrups or suspensions, for example solutions containing from about 0.2% to about 20% by weight of the active ingredients herein described, the balance being sugar and mixture of ethanol, water, glycerol and propylene glycol. Optionally such liquid formulations may contain colouring agents, flavouring agents, saccharine and carboxymethyl-cellulose as a thickening agent or other excipients known to a person skilled in the art.

Solutions for parenteral applications by injection can be prepared in an aqueous solution of a water-soluble pharmaceutically acceptable salt of the active substances, preferably in a concentration of from about 0.5% to about 10% by weight. Formulations for injection may be presented in unit dosage form, e.g. in ampules or in multi-dose containers, with an added preservative. The compositions may take such forms as suspensions, solutions or emulsions in oily or aqueous vehicles, and may contain formulating agents such as suspending, stabilizing and/or dispersing agents. Alternatively, the active ingredient may be in powder form for reconstitution with a suitable vehicle, e.g. sterile pyrogert-free water, before use.

The dosage amount of a combination of a 5-HT₄ agonist (or a pharmaceutically acceptable salt thereof) and an SSRI (or a pharmaceutically acceptable salt thereof), required to produce the therapeutic effect will, of course, vary and is ultimately at the discretion of the medical practitioner. As used herein the term “therapeutic effect” is the amount or dose of each component, the SSRI and the 5-HT₄ agonist, that provide faster onset of the therapeutic effect of the SSRI than when the SSRI is administered alone. The factors to be considered include: the route of administration and nature of the formulation; the subject's body weight, age and general condition; the nature and severity of the disease or disorder to be treated; the response of the subject or individual or subject; the properties of the drugs in combination as determined in clinical trials; the bioavailability characteristics of the preparation administered; the dose regimen selected; the use of concomitant medication; and other relevant circumstances. Dosage guidelines for some of the drugs can already be found in literature.

Toxicity and therapeutic efficacy of the compositions of the invention can be determined by standard pharmaceutical procedures in experimental animals, e.g., by determining the LD₅₀ (the dose lethal to 50% of the population) and the ED₅₀ (the dose therapeutically effective in 50% of the population). The dose ratio between therapeutic and toxic effects is the therapeutic index and it can be expressed as the ratio ED₅₀/LD₅₀. Compositions that exhibit large therapeutic indices are preferred.

The combination therapy of the present invention is carried out by administering the SSRI together with the 5-HT₄ agonist in a manner that provides effective levels of the two active ingredients in the body at the same time. All of the compounds concerned are orally available and are normally administered orally, and so oral administration of the adjunctive combination is preferred. They may be administered together, in a single dosage form, or may be administered separately. However, oral administration is not the only route or even the only preferred route. The compounds may be also be administered topically, parenterally, or mucosally (e.g., buccally, by inhalation, or rectally) in dosage unit formulations containing conventional non-toxic pharmaceutically acceptable carriers. It is usually desirable to use the oral route. The compounds may be administered orally in the form of a capsule, a tablet, or the like, or as a semi-solid or liquid formulation.

Preferred combination formulations are citalopram, fluoxetine, fluvoxamine or paroxetine as the SSRI and prucalopride or RS 67333 as the 5-HT₄ agonist.

Citalopram is disclosed, for example, in U.S. Pat. No. 4,136,193 as a serotonin reuptake inhibitor. Its pharmacology was disclosed by Christensen et al., Eur. J. Pharmacol. 41, 153 (1977), and reports of its clinical effectiveness in depression may be found in Dufour et al., Int. Clin. Psychopharmacol. 2, 225 (1987).

Fluoxetine is marketed in the hydrochloride salt form, and as the racemic mixture of its two enantiomers. U.S. Pat. No. 4,314,081 is an early reference on the compound.

Fluvoxamine is taught, for example, by U.S. Pat. No. 4,085,225. Scientific articles about the drug have been published by Claassen et al., Brit. J. Pharmacol. 60, 505 (1977); and De Wilde et al., J. Affective Disord. 4, 249 (1982); and Benfield et al., Drugs 32, 313 (1986).

Paroxetine may be found, for example, in U.S. Pat. Nos. 3,912,743 and 4,007,196. Reports of the drug's activity are in Lassen, Eur. J. Pharmacol. 47, 351 (1978); Hassan et al., Brit. J. Clin. Pharmacol. 19, 705 (1985); Laursen et al., Acta Psychiat. Scand. 71, 249 (1985); and Battegay et al., Neuropsychobiology 13, 31 (1985).

The present invention is further described by the following examples. The examples are provided solely to illustrate the invention by reference to specific embodiments. These examples, while illustrating certain specific aspects of the invention, do not portray the limitations or circumscribe the scope of the disclosed invention. Indeed, many modifications and variations of the invention will be apparent to those skilled in the art upon reading this specification and can be made without departing from its spirit and scope. The invention is therefore to be limited only by the terms of the appended claims along with the full scope of equivalents to which the claims are entitled.

### Example 1

### 5-HT₄ Receptors in the Control of the Raphé 5-HT Neuronal Firing Activity: Acute Studies

Biochemical studies have reported that the administration of 5-HT₄ agonists enhance the hippocampal release of serotonin (5-HT) in rodents, 5-HT₄ antagonists inducing an opposite effect. Despite the potential interest of such results for improved treatments of depression, little is none concerning the role of 5-HT₄ receptors in the control of the raphé-hippocampal 5-HT transmission. In the first part of a study, the 5-HT₄ agonists cisapride and prucalopride, as well as the selective 5-HT₄ antagonist GR 125487, were administered to anaesthetized rats and their effect on dorsal raphé 5-HT neuron firing rate was monitored in vivo by extracellular single-unit recording. These results indicated that 5-HT₄ receptors exert an excitatory tonus on the firing of certain 5-HT neurons, contributing to maintain their basal discharge at a high frequency. This tonus is not saturating, given that high-frequency 5-HT neurons can also be stimulated by 5-HT₄ agonists.

These studies, which provide evidence for the involvement of 5-HT₄ receptors in the control of the dorsal raphé nucleus 5-HT neuronal activity, are described in detail in Lucas, G. and Debonnel, G., (2002), 5-HT₄ receptors exert a frequency related facilitatory control on dorsal raphé nucleus 5-HT neuronal activity, European Journal of Neuroscience, Vol 16, pp. 817-822, incorporated herein by reference.

### Example 2

### 5-HT₄ Receptors in the Control of the Raphé 5-HT Neuronal Firing Activity: Subacute and Chronic Studies

In a second part of the study, the selective 5-HT₄ agonists prucalopride (Briejer et al., 2001) and RS 67333 (Eglen et al., 1995) were administered either subacutely (i.e. 30 min before the beginning of recordings), or continuously through the use of osmotic minipumps during 3 or 21 days. The selective 5-HT₄ antagonist GR 125487 (Gale et al., 1994) was also used to confirm the involvement of 5-HT₄ receptors in the effects of the agonists. Successive single-cell extracellular recording tracks were then performed along the DRN of anaesthetized rats. This set of experiments was performed in order to globally assess the effect of such treatments on the mean activity of 5-HT neurons. The obtained results indicate that both agonists enhanced the mean firing rate of 5-HT neurons. Their effect was also assessed after 3 days of treatment wherein a similar increase of 5-HT neuronal activity was observed, which was later suppressed upon administration of GR 125487 before recording. Finally, this action of the agonists is still present after 3 weeks, indicating that 5-HT₄ receptors responsible for this effect do not desensitize.

Animals. Experiments were carried out in male Sprague-Dawley rats (Charles River, St-Constant, Québec, Canada), weighing 270 to 300 g and kept under standard laboratory conditions. Experiments were done in conformity with the Canadian Guide to the Care and Use of Experimental Animals (Vol. 1, 2nd edition, 1993).

Drugs. The following compounds were used: GR 125487 sulphamate, prucalopride monohydrochloride (gifts from Glaxo and Janssen laboratories, respectively), RS 67333 hydrochloride and 5-HT hydrochloride (Tocris Cookson Inc., Ellisville, Mo.).

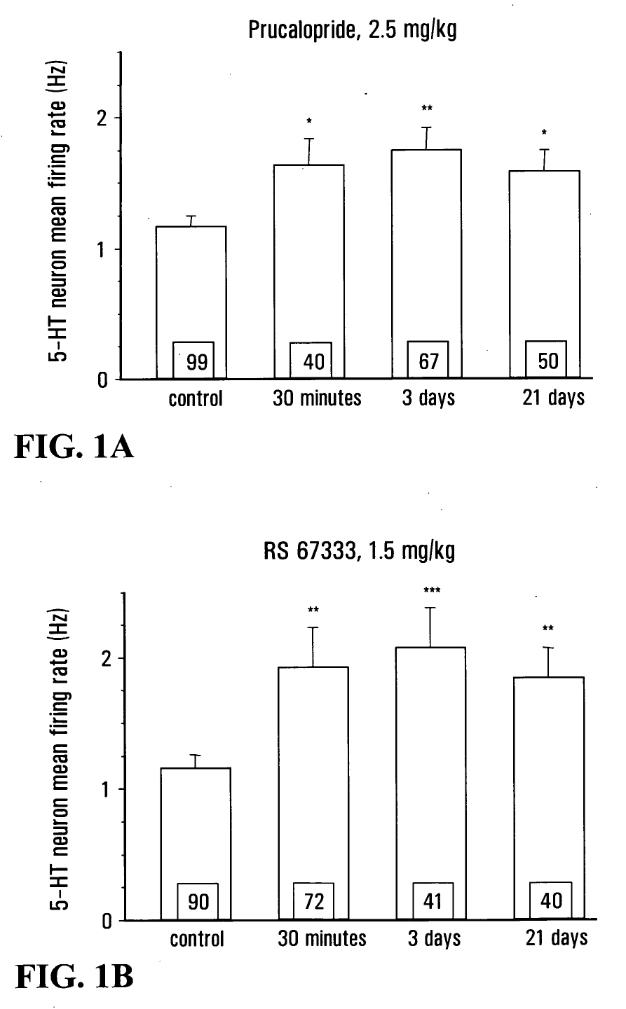

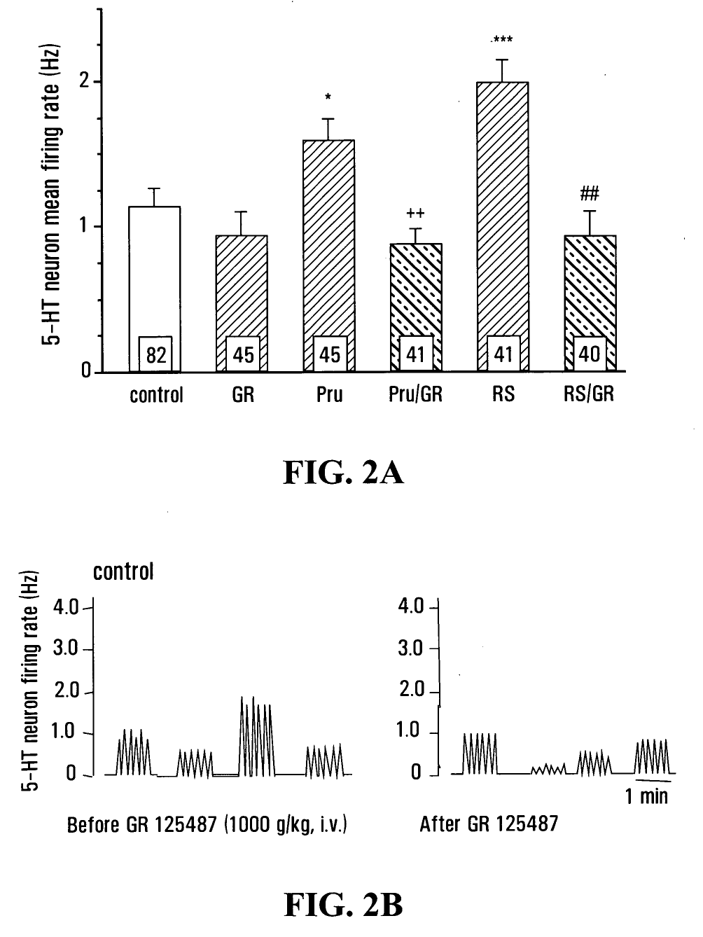

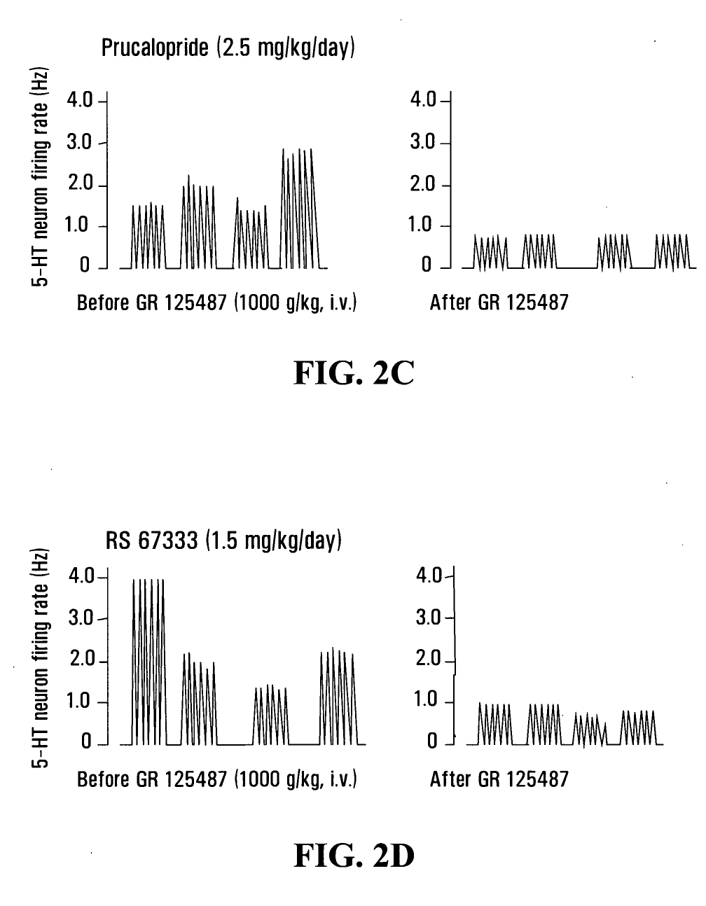

Pharmacological treatments. All the 3 different 5-HT₄ pharmacological agents were diluted in a solution of physiological saline (NaCl 0.9%), and administered in a dose range known to induce significant binding on 5-HT₄ sites (Lucas and Debonnel, 2002; Lamirault and Simon, 2001; Porras et al., 2002). In subacute experiments, prucalopride, RS 67333 or their vehicle were administered i.p. 30 min. before the beginning of recordings. For chronic treatments (both 3 and 21 days), prucalopride, RS 67333 or their vehicle were delivered through osmotic minipumps (Alza, Palo Alto, Calif.) inserted subcutaneously. Electrophysiological recordings were performed with the minipumps in place. Results of vehicle-treated (i.p. and via minipumps) animals were not statistically different from each other, and data were pooled and randomized to constitute the control groups displayed in FIGS. 1 and 2. In a separate set of experiments, the 5-HT₄ antagonist GR 125487 was tested in animals treated for 3 days with either prucalopride or RS 67333. Two DRN tracks were first performed, then GR 125487 was administered i.v. (via a lateral vein of the tail) and 2-3 additional tracks were performed, starting 30 min. after the injection. All drug dosages refer to the free base.

Extracellular recordings of DRN 5-HT neurons. Recordings were performed using single-barreled glass micropipettes. Electrodes were filled with a 2 M NaCl solution saturated with Fast Green FCF, resulting in an impedance of 2-5 MΩ. Rats were anaesthetized with chloral hydrate (400 mg/kg, i.p.) and placed in a stereotaxic frame. A burr hole was drilled on the midline 1 mm anterior to lambda. DRN 5-HT neurons were encountered over a distance of 1 mm starting immediately below the ventral border of the Sylvius aqueduct. These neurons were identified using the classical criteria: a slow (0.5-2.5 Hz) and regular firing rate and long-duration (0.8-1.2 ms) positive action potentials (Aghajanian, 1978). In each animal, four to five successive descents were performed along the DRN, and a total of 10-16 cells was recorded. At the end of the experiments, a 25 μA cathodal current was passed through the recording electrode to leave a Fast Green deposit at the recording site. Animals were sacrificed with an i.v. overdose of chloral hydrate, and the brain removed. The site of recording was verified under microscope immediately after experiments.

Statistical analysis. In each experimental group, data were expressed as means±S.E.M. of all the recorded neurons, unless otherwise specified. The effects of the different 5-HT₄ agonist administrations on the firing activity of DRN 5-HT neurons was assessed using one-way ANOVAs, followed by the post-hoc Dunnett's test when significant. The influence of GR 125487 on the effect of a 3-day treatment with prucalopride or RS 67333 was assessed using two-way ANOVAs followed by the post-hoc Tukey's test. In all cases, p<0.05 was chosen as the criterion of significance.

Effect of Subacute and Chronic Treatments with 5-HT₄ Agonists on DRN 5-HT Mean Neuronal Firing Rate.

The i.p. administration of both prucalopride and RS 67333 induced an enhancement of DRN 5-HT neuronal mean firing rate, already observable 30 min after the injection. Such a kinetic is compatible with previous in vivo studies conducted with the same compounds (Lamirault and Simon, 2001; Porras et al., 2002). In addition, the action of the agonists remained almost the same after a continuous treatment of 3 days. As shown in FIG. 1, the administration of both prucalopride (2.5 mg/kg) and RS 67333 (1.5 mg/kg) induced an increase of DRN 5-HT activity [one-way ANOVAs, F(3, 252)=4.51, p<0.01 and F(3, 240)=6.5, p<0.001 for prucalopride and RS 67333, respectively]. More precisely, the subacute administration of prucalopride elevated 5-HT neuron firing rate by 40% with respect to controls (Dunnett's test, p<0.05 vs control, FIG. 1A); in the same experimental condition, the effect of RS 67333 was more pro-eminent, reaching 66% above basal values (Dunnett's test, p<0.01 vs control, FIG. 1B). The effect of these two compounds was remarkably stable over time, as illustrated by the results of short- and long-term chronic treatments. Indeed, a 3- and 21-day administration of prucalopride resulted in a +50 and +36% enhancement of DRN 5-HT neuronal activity, respectively (Dunnett's test, p<0.01 and p<0.05 vs control, respectively; FIG. 1A). Similarly, a 3-day treatment with RS 67333 elevated 5-HT neuron mean firing rate by 79%, and a 21-day treatment by 59% (Dunnett's test, p<0.001 and p<0.01 vs control, respectively; FIG. 1B).

Influence of the 5-HT₄ Antagonist GR 125487 on the Effect of 3-Day Treatment with 5-HT₄ Agonists.

The involvement of 5-HT₄ receptors in these latter effects was confirmed by their complete blockade following an i.v. administration of the selective 5-HT₄ antagonist GR 125487. FIG. 2 illustrates the effect of an acute, i.v. administration of GR 125487 on the increase of DRN 5-HT neuron firing rate induced by 3-day treatments with either prucalopride or RS 67333. The facilitory effect of both 5-HT₄ agonists was totally suppressed by GR 125487 [two-way ANOVAs, F(1, 210)=8.6, p<0.01 and F(1, 205)=9.7, p<0.01 for prucalopride and RS 67333, respectively; FIG. 2A]. GR 125487 induced on its own a small, non statistically significant (Tukey's test, n.s. vs control) reduction of 5-HT neuronal activity. In the presence of prucalopride and RS 67333, the effect of GR 125487 was very similar, indicating a total blockade of the elevation induced by these latter compounds (Tukey's test, p<0.01 vs prucalopride and RS 67333 alone, and n.s. vs control, for the prucalopride+GR 125487 and RS 67333+GR 125487 groups, respectively). The comparison of the recording samples in the left panels of FIGS. 2B, 2C and 2D further illustrates the effect of a 3-day treatment with the 5-HT₄ agonists on DRN 5-HT neuron firing rate. In both the prucalopride and RS 67333 groups, neurons discharging over 2.0 Hz were frequently observed, at variance with control animals. However, after the injection of GR 125487, the patterns of neuronal firing along the descents were similar in all 3 groups, with a majority of neurons presenting an activity in the 1.0 Hz range (FIGS. 2B, 2C and 2D, right panels).

### Discussion

The results obtained with the agonists indicate that, even if only one-half of neurons are affected by the acute administration of 5-HT₄ agents (Lucas and Debonnel, 2002), the 5-HT₄-mediated excitatory control is significant when considering the whole DRN. Hence, given that the DRN contains a large majority of the 5-HT soma found in the brain (Descarries et al., 1982), it appears possible to globally influence the central 5-HT function by acting through 5-HT₄ transmission. Furthermore, 5-HT₄ receptors responsible for this control do not seem to desensitize, as the effect of both agonists is unchanged after a long-term (21 days) treatment. Obviously, this remarkable stability over time supports the potential use of 5-HT₄ agonists for the treatment of depression, as, beyond the case of SSRIs and their delayed onset of action, it is rarely observed with other drugs known to increase central 5-HT transmission. For instance, whereas, in acute conditions, the new antidepressant mirtazapine increases 5-HT neuron firing rate (through an indirect mechanism involving the release of norepinephrine) (Haddjeri et al., 1996), this effect is no longer present after a 2-day treatment (Besson et al., 2000). It appears, therefore, that 5-HT₄ receptors are able to exert a potent influence on DRN 5-HT function.

### Example 3

### Assessment of the Effect of the 5-HT₄ Receptor Agonists Prucalopride and RS 67333, Alone or in Combination with Classical Antidepressants, in the Forced Swimming Test (FST) Paradigm

The “forced swimming test” (FST) is a behavioural test for rodents, which predicts the efficacy of antidepressant drugs (Porsolt et al., 1977; Lucki, 1997). The FST model is an accepted standard for detecting and comparing the antidepressant activity of different classes of antidepression compounds for which there is a good correlation with human antidepression activity. The widespread use of this model is largely a result of its ease of use, reliability across laboratories and ability to detect a broad spectrum of antidepressant agents. The test is based on the observation that rats, following initial escape-oriented movements (climbing and swimming), develop an immobile posture when placed in an inescapable cylinder of water. If they are replaced in the same testing apparatus 24 h later, they resume this immobile posture quickly. The immobility is thought to reflect either a failure of persistence in escape-directed behavior (i.e. behavioral despair) or the development of passive behavior that disengages the animal from active forms of coping with stressful stimuli. If antidepressant treatments are given between the two exposures, the subjects will actively persist engaging in escape-directed behaviors for longer periods of time than after vehicle treatment. This will be reflected by a reduction of immobility time, and by an increase of climbing attempts. Details of this test method can be found, for example, in Willner (1984) in Psychopharmacology 83:1-16, Borsini and Meli (1988) in Psychopharmacology 94:147-160 or Cervo et al. (1992) in Neuropharmacology 31:331-335. The combination effect of a particular 5-HT₄ agonist and a SSRI may be evaluated using the FST model.

Although a presynaptic component has been also proposed (Cervo and Samanin, 1987; Cervo and Samanin, 1991), it appears that postsynaptic 5-HT_{1A} receptors play a key role in the immobility effects of antidepressants (Wieland and Lucki, 1990; Luscombe et al., 1993; Detke et al., 1995; Lucki et al., 1994). Therefore, the inventors assessed the effect of the selective 5-HT₄ receptor agonists prucalopride and RS 67333, alone or in combination with the classical antidepressants, in the FST paradigm.

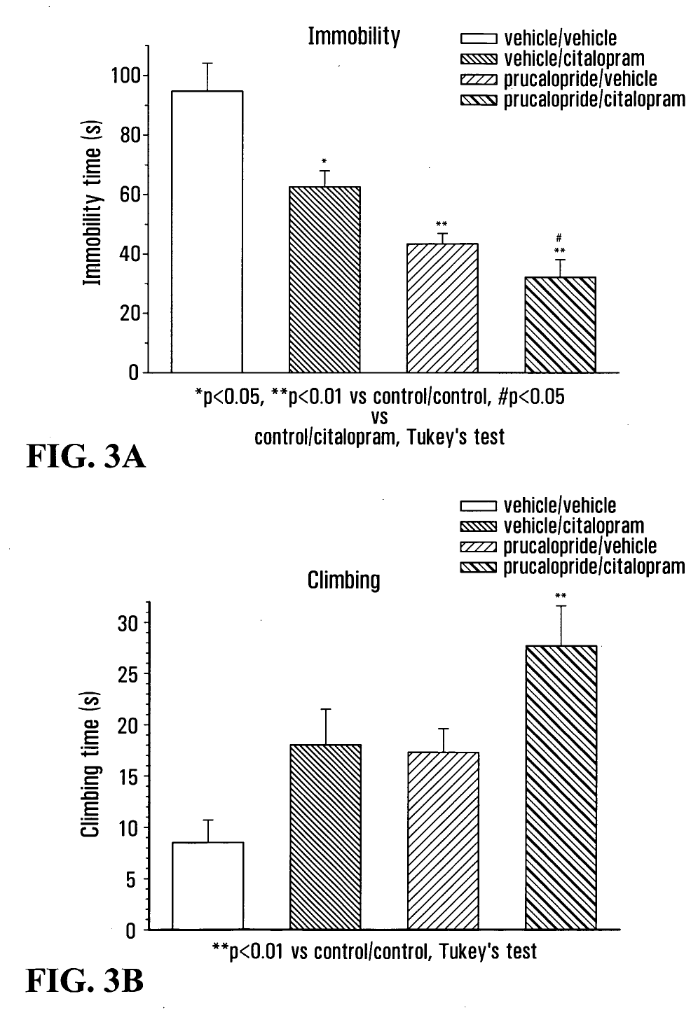

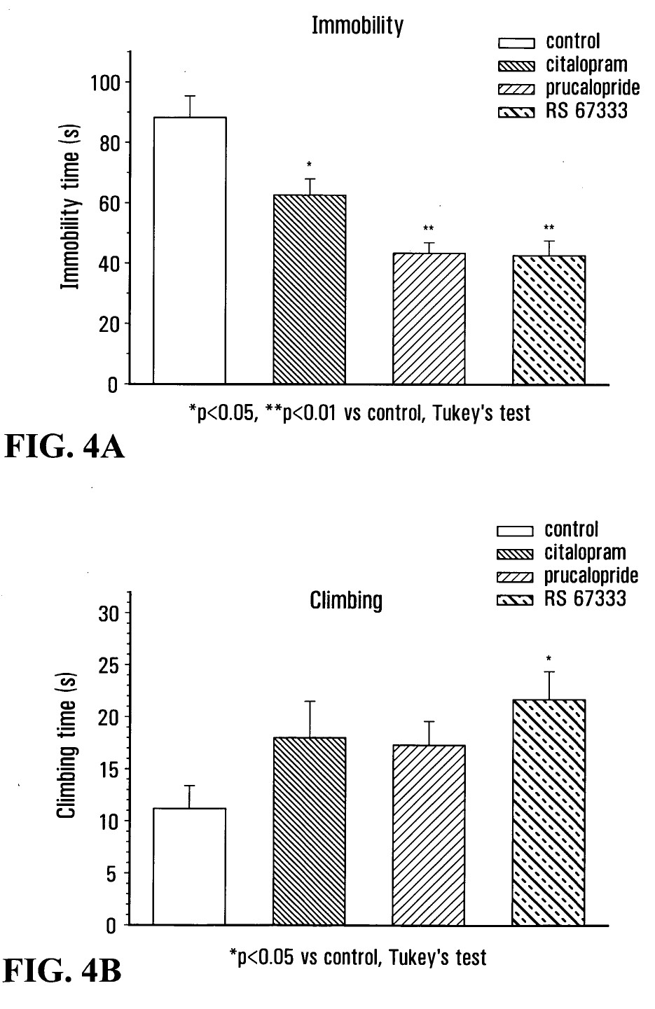

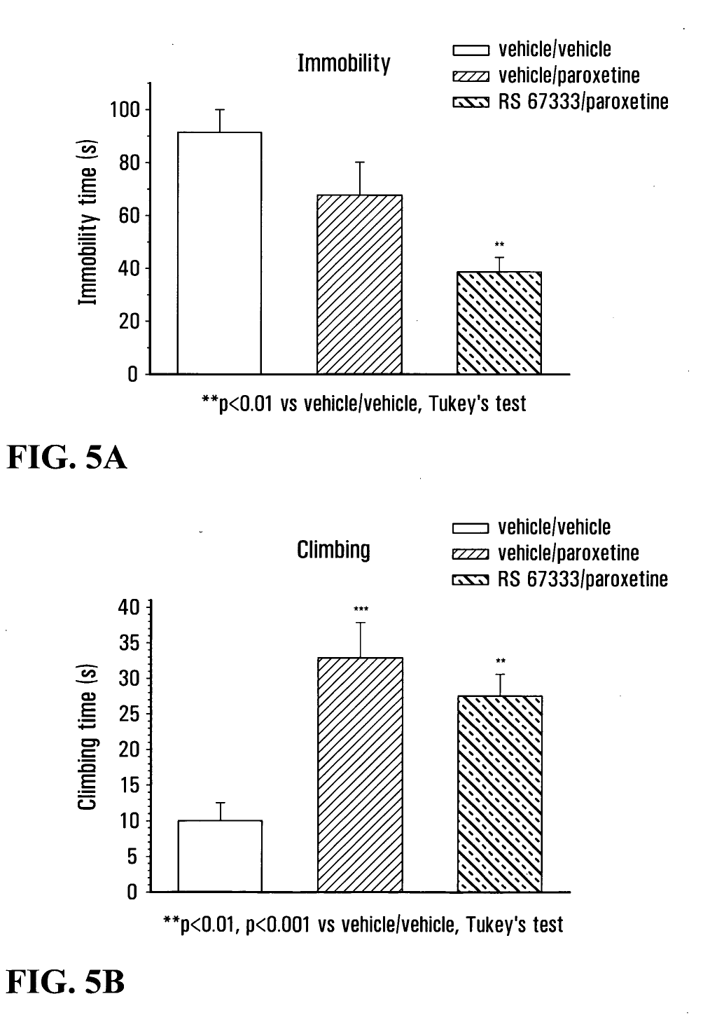

Panel A of FIG. 3 shows the effect of citalopram and prucalopride, alone or in combination, on the total time of immobility behavior evaluated in the FST. A one-way ANOVA indicates that there was a strong difference between the 4 groups [F(3, 24)=18.4, p<0.001]. The post-hoc Tukey's test reported a small, but significant effect of citalopram alone (−34%). In contrast, prucalopride alone displayed a more robust effect (−54%), and the combination of prucalopride and citalopram divided the time of immobility by 4 (−66%). A very similar effect was observed with RS 67333 alone (FIG. 4). The two-way ANOVA, performed with prucalopride and citalopram as the two factors, was not significant [F(1, 24)=2.6, n.s.], indicating that the effects of prucalopride and citalopram were statistically additive (FIG. 3). Again, the combination of RS 67333 with the other SSRI paroxetine resulted in a similar reduction of immobility time (−62%, FIG. 5).

In panel B of FIG. 3, the inventors have illustrated the effect of prucalopride and citalopram on the climbing behavior of animals. Again, the one-way ANOVA revealed a significant difference between the 4 groups [F(3, 24)=6.4, p<0.01], which was related to a significant increase of climbing attempts in the prucalopride/citalopram group (+226%). Also, the two-way ANOVA indicated that the effects of prucalopride and citalopram were statistically additive [F(1, 24)=0.02, n.s.].

The reduction of immobility time in the FST is a very good predictor of antidepressant activity (Porsolt et al., 1977; Lucki, 1994). When administered only once before the testing session, classical antidepressants of the SSRI class, such as citalopram, display only a low efficiency (about −30%) (Sanchez et al., 2003), or no significant effect (Tatarczynska et al., 2002). These results are therefore in agreement with previous studies conducted with this SSRI. Much more interestingly, the administration of the 5-HT₄ agonist prucalopride led to a strong reduction in the time of immobility. So far, all the molecules which have been effective in this paradigm (i.e. MAO, tricyclics, SSRIs, SNRIs, NASSAs) have also displayed antidepressant effects in clinical trials.

Although prucalopride and RS 67333 constitute putative antidepressants on their own, they are also more efficient than citalopram or paroxetine after a single administration, suggesting that they may have a faster onset of action, as an antidepressant, than SSRIs (Tatarczynska et al., 2002). Indeed, 5-HT₄ agonists directly activate the 5-HT neuron firing rate (Lucas and Debonnel, 2002), a mechanism that does not require a time-dependent desensitization of 5-HT_{1A} autoreceptors to enhance 5-HT neurotransmission. Such a direct facilitation of 5-HT activity has been proposed to underlie a faster onset of action in antidepressant therapy (Blier, 2001).

Furthermore, the combination of prucalopride or RS 67333 and classical SSRIs was even more effective than the 5-HT₄ agonists alone in reducing the time of immobility. The additivity of the effects of these two types of compounds confirm that they acted through distinct, independent, mechanisms in increasing 5-HT neurotransmission. Obviously, this result opens even more exciting avenues in the field of depression. A bi-therapy, based on the concomitant administration of a classical SSRI with a “booster” of 5-HT function, may constitute the most efficient way to achieve both a more rapid onset of antidepressant action, and a clinical improvement in patients resistant to antidepressant monotherapy (Blier, 2001).

Experiments were carried out in male Sprague-Dawley rats (Charles River, St Constant, Québec). We used the FST as previously described (Porsolt et al., 1977). Briefly, the rats experienced a pretest session followed 24 hours later by a test session. For both the pretest and the test sessions, conducted under low illumination (12 lx), the rats were placed in a plastic cylindrical tank (50 cm high by 20 cm in diameter) filled with water at 240±2° C., with a depth of 40 cm, for which the hind limbs could not reach the tank floor. In all experiments, the pretest was carried out for 15 min and the test for 5 min in the same tank but only the last 4 min were analyzed. Following either pretest or test sessions, rats were dried with a towel and kept warm for 30 min before returning in their home cage. A digital camera recorded on line animal's behavior during the FST, and both time of immobility and climbing attempts were counted. Citalopram hydrobromide (10 mg/kg, i.p.) or paroxetine (10 mg/kg, i.p.) and prucalopride hydrochloride (2.5 mg/kg, i.p.) or RS 67333 hydrochloride (1.5 mg/kg, i.p.) were administered simultaneously 30 minutes before the test session. Animals were divided into 4 groups: vehicle/vehicle, 5-HT₄ agonist/vehicle, vehicle/SSRI and 5-HT₄ agonist/SSRI. For each compound, distilled water was used as the vehicle. All drug dosages refer to the free base.

### Example 4

### Dosage Effect of the 5-HT₄ Receptor Agonists Prucalopride and RS 67333, Alone or in Combination with Classical Antidepressants, in the Forced Swimming Test (FST) Paradigm

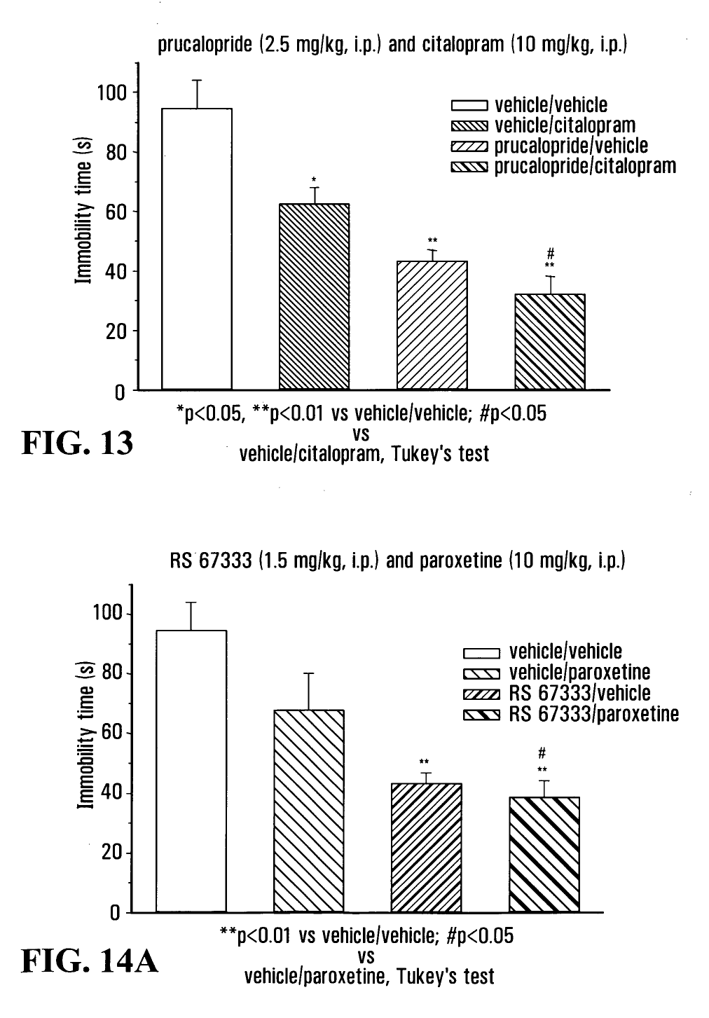

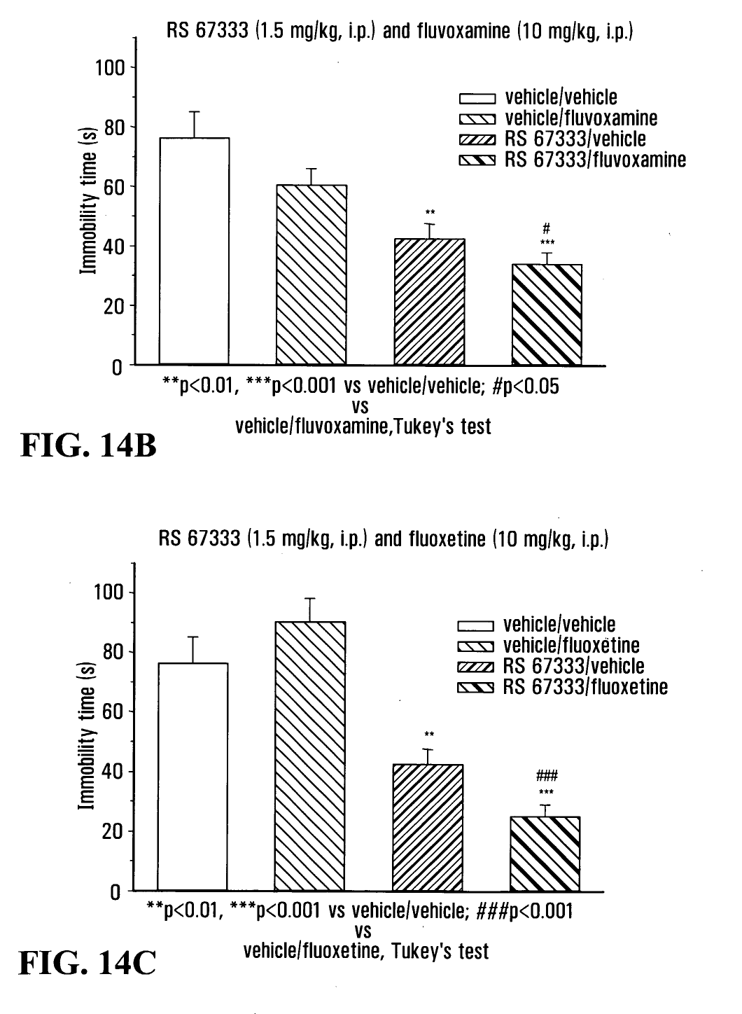

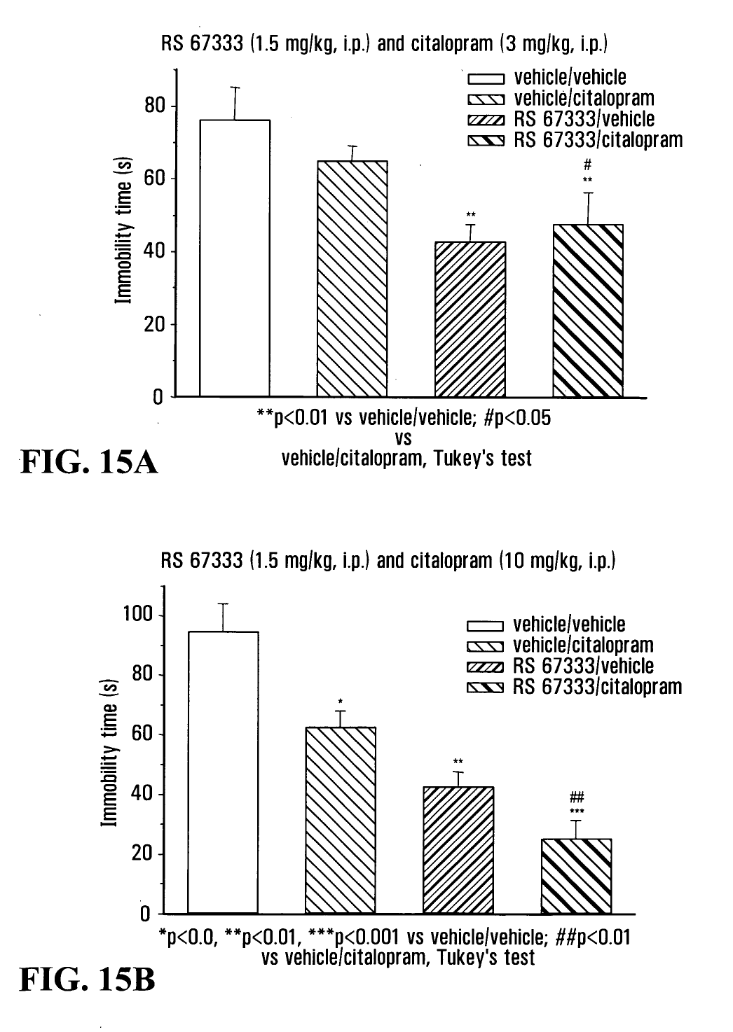

Referring to FIGS. 13, 14A to 14C and 15B, the results show that citalopram, at the dose of 10 mg/kg, induced a slight, but significant, reduction of immobility. This is in agreement with earlier studies, showing a similar modest effect for this dose (which, however, did not reach statistical significance in all the reports). Our results are also in keeping with several findings from other experimental models, indicating that 10 mg/kg constitutes the minimal dose for citalopram to become effective. Thus, citalopram had no effect on immobility when administered at 3 mg/kg (see FIG. 15A). However, the co-administration of 3 mg/kg citalopram with RS 67333 induced a strong reduction of immobility. This effect was much more important than the one observed when citalopram was given alone at 10 mg/kg (see FIG. 15B). Our results therefore suggest that the adjunction of a 5-HT₄ agonist to a SSRI may allow to reduce the dose of the latter, with an even greater efficacy.

### Example 5

### Effect of the 5-HT₄ Receptor Agonist Prucalopride on the Inhibition of DRN 5-HT Neuron Firing Rate Induced by the SSRIs Fluvoxamine and Citalopram

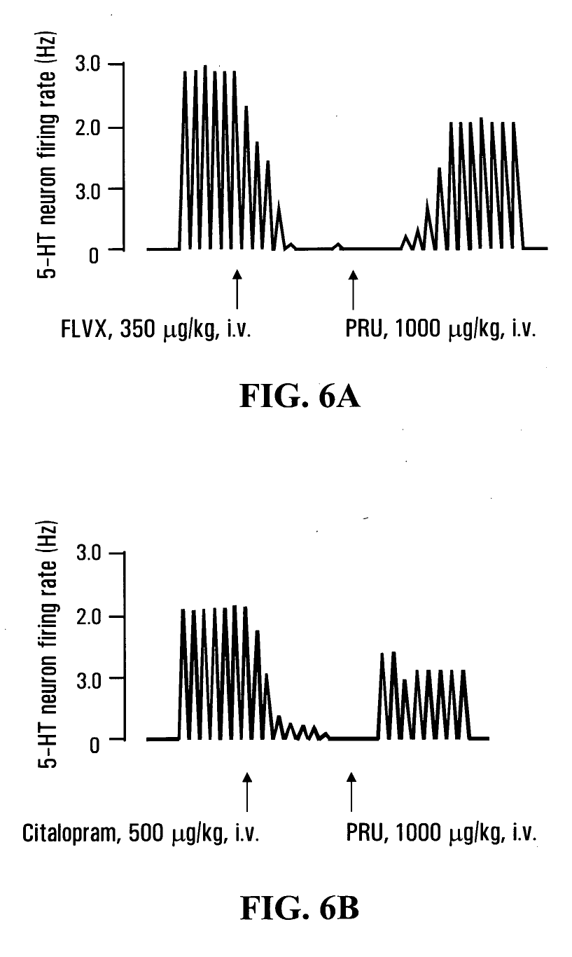

In FIGS. 6A and 6B, the firing activity of the 5-HT neuron is suppressed following the acute intravenous administration of the antidepressant SSRIs fluvoxamine and citalopram. This suppression of the firing activity is due to the activation of the 5-HT_{1A} autoreceptors, subsequent to the increase of the concentration of endogenous 5-HT, following the blockade of the 5-HT reuptake sites. An acute administration of prucalopride is able to partially reverse this effect in about one-half of 5-HT neurons. This result strengthens the conclusion that the combination of a 5-HT4 agonist with an SSRIs proves useful to counteract the initial inhibition of 5-HT activity induced by the latter, therefore confirming the putative interest of 5-HT4 agonists as “boosters” of antidepressant efficacy.

### Example 6

### 5-HT₄ Receptor Agonists as Putative Antidepressants with Rapid Onset of Action

Current antidepressants are clinically effective only after several weeks of administration. Here Applicants show that 3-day a regimen with 5-HT₄ agonists modify rat brain parameters considered as key markers of antidepressant action, but observed only after 2-3 week treatments with classical molecules. These changes include a desensitization of DRN 5-HT_{1A} autoreceptors, an increased tonus on hippocampal postsynaptic 5-HT_{1A} receptors, as well as enhanced phosphorylation of the cAMP response element-binding protein (CREB) and neurogenesis in the hippocampus. Moreover, 5-HT₄ agonists strongly reduce immobility in the FST. Together, these findings point out 5-HT₄ receptor agonists as a putative new class of antidepressants, with a rapid onset of action.

Animals. Experiments were carried out in male Sprague-Dawley rats (Charles River, St-Constant, Québec, Canada, and Harlan, Gannat, France) weighing 250-300 g, which were kept under standard laboratory conditions (12:12 light-dark cycle with free access to food and water).

Different rats were used for the FST and the locomotor activity responses. All animals were handled according to the guidelines approved by the Faculty ethical committees of our institutions.

Drugs and Chemicals. The following compounds were used: GR 125487 sulphamate, prucalopride monohydrochloride, citalopram hydrobromide (gifts from Glaxo, Janssen and Lundbeck laboratories, respectively), RS 67333 hydrochloride (Tocris Cookson Inc., Ellisville, Mo., USA), WAY 100635 hydrochloride (Research Biochemicals, Natick, Mass., USA), pCPA methyl ester hydrochloride, BrdU (Sigma-Aldrich Canada, Oakville, Ontario, Canada). All compounds were diluted in distilled water. All drug dosages refer to the free base. For chronic treatments, prucalopride (2.5 mg/kg/day), RS 67333 (1.5 mg/kg/day) or the vehicle were delivered through osmotic minipumps (Alza, Palo Alto, Calif., USA), inserted subcutaneously in the region of the back under short-duration (≦5 min) halothane anesthesia. The 5-HT depleter PCPA was administered once daily at the dose of 150 mg/kg, i.p., 72, 48 and 24 h before the recordings. BrdU treatments consisted of two administrations per day (50 mg/kg, i.p. each, 8 h interval), starting from the day of minipump insertion until day 2 post-surgery.

Extracellular recordings of DRN 5-HT neurons. Recordings were performed using single-barreled glass micropipettes. Electrodes were filled with a 2 M NaCl solution saturated with Fast Green FCF, resulting in an impedance of 2-5 MΩ. Rats were anaesthetized with chloral hydrate (400 mg/kg, i.p.) and placed in a stereotaxic frame. A burr hole was drilled on the midline 1 mm anterior to lambda. DRN 5-HT neurons were encountered over a distance of 1 mm starting immediately below the ventral border of the Sylvius aqueduct. These neurons were identified using the classical criteria: a slow (0.5-2.5 Hz) and regular firing rate and long-duration (0.8-1.2 ms) positive action potentials (Aghajanian, 1978). At the end of the experiments, a 25 μA cathodal current was passed through the recording electrode to leave a Fast Green deposit at the recording site. Animals were sacrificed with an i.v. overdose of chloral hydrate, and the brain removed. The site of recording was verified under microscope immediately after experiments.

Microiontophoresis and extracellular recordings from hippocampal CA₃ pyramidal neurons. Recording and microiontophoresis were performed with five-barreled glass micropipettes broken back to 8-12 μm under microscope control. The central barrel was filled with the same solution as in the DRN and was used for extracellular unitary recordings. Pyramidal neurons were identified by their large amplitude (0.5-1.2 mV) and long-duration (0.8-1.2 ms) simple spikes alternating with complex spike discharges (Kandel and Spencer, 1961). The side barrels contained the following solutions: quisqualate (1.5 mM in 200 mM NaCl, pH 8), WAY 100635 (15 mM in 200 mM NaCl, pH 4), and 2 M NaCl used for automatic current balancing. Rats were mounted in the stereotaxic apparatus and the micropipettes were lowered at 4.2 mm lateral and 4.2 anterior to lambda into the CA₃ sub-region of the dorsal hippocampus.

Assessment of CREB and pCREB immunoreactivities. Following decapitation (performed under halothane anesthesia) rat brains were dissected on ice cold artificial cerebrospinal fluid (125 mM NaCl, 2.4 mM KCl, 0.83 mM MgCl2, 1.1 mM CaCl2, 0.5 mM KH2PO4, 0.5 mM NaSO4, 27 mM NaHCO3, 10 mM glucose, 10 mM Hepes pH 7.4). Isolated hippocampi (1-2 mg wet tissue/100 μl) were homogenized in solubilization buffer containing 20 mM Hepes pH 7.9, 0.4 M NaCl, 20% (v/v) glycerol, 1% (v/v) Nonidet P-40, 5 mM MgCl2, 0.5 mM EDTA, 0.1 mM EGTA, 1 mM phenylmethanesulfonyl fluoride, 1 μM okadaic acid, 5 mM dithiothreitol, 5 μg/ml leupeptin, 5 μg/ml soybean trypsin inhibitor, and 10 μg/ml benzamidine by means of a dounce homogenator. Homogenates were then adjusted to a concentration of 2 mg protein/ml and incubated on ice for 30 min, after which they were centrifuged for additional 30 min at 15,000 g. The supernant was discarded and SDS sample buffer was added to the pellet for posterior immunoblot analysis. For detection of CREB activation samples were sonicated and then boiled for 5 min before loading for SDS-PAGE that was performed as previously described (Piñeyro et al., 2001) using a 4% stacking gel and 10% separating gel. Proteins resolved in SDS-PAGE were then transferred from gels onto nitrocellulose (50 mA, 16 h, Bio-Rad Mini-Trans Blot apparatus) and PCREB detected by probing membranes with anti-pCREB monoclonal antibody (1B6) from Cell Signaling Technology (1:1000). Total CREB contents was determined after stripping by using 1:1000 dilution of anti-CREB antibody (Cell Signaling Technology). Secondary antimouse (1:5000; Sigma) or antirabbit (1:40000; Amersham) horseradish-conjugated antibodies and enhanced chemiluminescence detection reagents (NEN Life Science Products) were used to reveal blotted proteins. Relative intensities of the labeled bands were analyzed by densitometric scanning using MCID (Imaging Research Inc) and CREB-activation was expressed as the ratio between PCREB and total CREB present in each sample.

Measurement of hippocampal neurogenesis. Animals were deeply anesthetized by using an overdose of sodium pentobarbital (75 mg/kg, i.p), and perfused transcardially with an initial wash of heparinized 0.9% saline (50-100 ml, 4° C.), followed by 4% paraformaldehyde in phosphate buffer (300 ml, 0.1M, pH 7.4, 4° C.). Brains were immersed for 48 hours in 30% phosphate-buffered sucrose solution (pH 7.4) and then cut in the coronal plane at 50 μm on a sliding freezing microtome. Free-floating sections were collected in phosphate-buffered saline (PBS, 0.1M, pH 7.4) as separate sets so that each set contained every sixth serial section. Selected adjacent free-floating sections were processed for double-labeling immunohistochemistry for BrdU (5′-bromodeoxyuridine) and Nissl staining, by using minor modifications of a previously published method (Soriano and Del Rio, 1991; Sadikot and Sasseville, 1997). Briefly, sections were incubated in 0.5% sodium borohydride dissolved in PBS for 20 minutes and rinsed twice in PBS. They were then incubated for 30 minutes in 1% Triton X-100 in PBS containing 0.03% hydrogen peroxide, followed by 1% dimethlysulfoxide (DMSO) in PBS for 10 minutes. Sections were immersed in 2N HCl in PBS for 60 minutes, and then neutralized by rinsing in sodium borate buffer (0.1M, pH 8.5) for 5 minutes. After brief washes in PBS (3×, 5 minutes each), they were pre-incubated in PBS containing 10% bovine serum albumin (BSA) and 0.3% Triton X-100 for 30 minutes, briefly rinsed in PBS, and then incubated for 14 to 16 hours in PBS containing anti-BrdU antibody (1:40, Becton-Dickinson, San Jose, Calif., USA) and 2% BSA (4° C.). After 3 brief rinses in PBS, sections were incubated in PBS containing secondary antibody (biotinylated antimouse IgG, 1:200, Vector, Burlinghame, Calif., USA) and 2% BSA. Following 3 brief rinses in PBS, sections were incubated for 1 hour in avidin-biotin complex (ABC, 1%, in PBS, Vector). Next, sections were briefly rinsed 3 times in PBS, and the immunohistochemical reaction product was revealed by incubating for 7-10 minutes in a solution containing 0.37 mg nickel ammonium sulfate, 25 mg 3.3′-diaminobenzidine tetrahydrochloride (DAB), and 2 μl of hydrogen peroxide (30%) dissolved in 100 ml of Tris buffer (0.05 M, pH 7.6). This nickel-enhanced DAB-based chromogen yields a blue-black reaction product. Sections were thoroughly rinsed in PBS, and then mounted out of distilled water on glass slides, air-dried, dehydrated in a 80% ethanol solution over night, then proceed with Cresyl Violet (CV) staining, cleared in Xylene Substitute (Shandon, Pittsburgh, Pa.), and coverslipped with Permount (Fisher, Fair Lawn, N.J., USA).

BrdU/CV double-labeled neurons were analyzed throughout the entire extend of the granular cell layer (GCL) of dentate gyrus including the subgranular zone (as defined by two cell-body wide zone at the edge of GCL) at one comparable coronal section (equivalent to Bregma—4.52 mm) (Paxinos and Watson, 1986) in four RS67333-treated and four control animals. The density of BrdU-positive neurons of treated and control animals were compared throughout the cross-section area of interest. Dentate gyrus distribution of BrdU-positive neurons was plotted using a system for image analysis. The left hemisphere was used for all quantitative analysis. The system consisted of a light microscope (BX40, Olympus, Japan) equipped with an X-Y movement-sensitive stage (BioPoint XYZ, LEP, Hawthorne, N.Y., USA), a Z-axis indicator (MT12 microcator, Heidenhain, Traunreut, Germany) and a video camera (DC200, DAGE, Michigan City, Ind., USA) coupled to a computer containing software for computer-assisted image analysis (Stereo Investigator, Microbrightfield Inc., Colchester, Vt., USA). The software allowed drawing of outlines of hippocampus sections at low (10× objective) magnification, and plotting of positions of single-(CV) or double-(BrdU/CV) labeled neurons evaluated at high (100× objective) magnification.

Behavioral experiments. We used the FST as previously described (Porsolt et al., 1977). Briefly, rats experienced a pre-test session followed 24 hours later by a test session. For both the pre-test and the test sessions, conducted under low illumination (12 lx), the animals were placed in a plastic cylindrical tank (50 cm high by 20 cm in diameter) filled with water at 240-±1° C., with a depth of 40 cm, for which the hind limbs could not reach the tank floor. In all experiments, the pre-test was carried out for 15 min and the test for 5 min in the same tank but the last 4 min only were analyzed. Prucalopride (2.5 mg/kg, i.p.) and RS 67333 (1.5 mg/kg, i.p.) were administered 30 min before the test session. Following either pre-test or test sessions, rats were dried with a towel and kept warm for 30 min before returning in their home cage. A camera coupled with a computer recorded on line animal behavior during the FST through a specialized digital interface (Videotrack, ViewPoint, Lyon, France). This interface underscored on line the subtraction of video frames. Immobility time in FST was derived from the number of frames (every 40 ms) being below a predefined threshold over FST duration. This threshold was preliminarily set up in order to obtain about 95% of the corresponding frames classified as immobile for a non-swimming rat in its water tank. The same threshold was kept constant for naive as well as treated animals.

Measurements of locomotion were also performed, in order to ensure that the decreased immobility or the increased active behaviours in the FST were not secondary to a non-specific increase in motor activity produced by the treatments. Thirty min after drug administration, rats were placed in activity cages in whom photoelectric cells were inserted allowing recordings of locomotor activity, i.e. quantification of the total number of activity counts (photocell beam breaks) during 10 min. session.

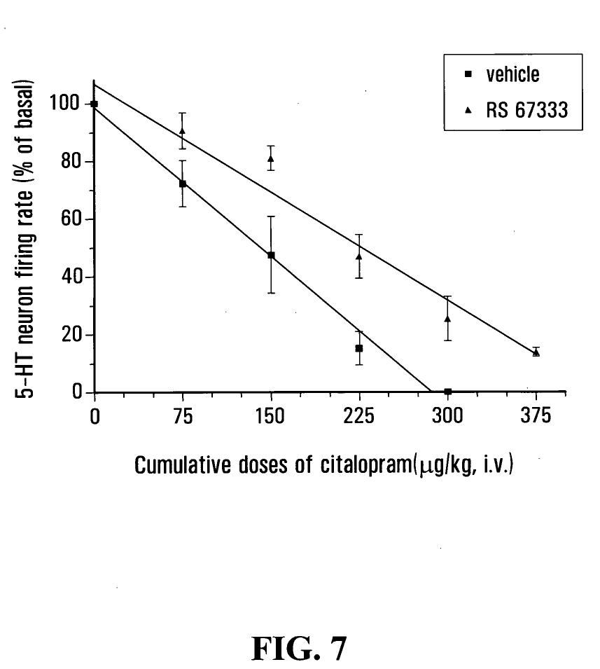

Statistical analysis. The comparison between the two slopes in FIG. 7 was performed by using the GraphPad Prism software (Version 3.03, GraphPad Software Inc.). All other statistics were performed by using one-way ANOVAs, followed by the Dunnett's test when multiple comparisons were necessary.

Effect of a 3-day treatment with a 5-HT₄ agonist on DRN 5-HT_{1A} autoreceptors sensitivity. As previously reported (Sanchez et al., 2003), the i.v. administration of the SSRI citalopram induced a dose-dependent inhibition of 5-HT neuronal firing rate in naïve animals, with an ED₅₀ of 130±30 μg/kg and an ED₁₀₀ of 280 μg/kg (FIG. 7). In rats treated with the selective 5-HT₄ agonist RS 67333 (Eglen et al., 1995) (1.5 mg/kg/day), these values were shifted to the right, reaching 226±26 and 430 μg/kg, respectively. A statistical comparison revealed that the slopes of the two dose-response curves were significantly different (−0.34±0.03 vs −0.25±0.02 for the vehicle and RS 67333 groups, respectively; F (1, 38)=6.6, p=0.014), confirming that the shift observed in the presence of RS 67333 was due to a decreased sensitivity to citalopram. It is important to mention that this shift was observed with a similar amplitude in all neurons recorded from the RS 67333 group, their basal firing rate being either high (≧2.0 Hz) or low (≦1.0 Hz) (see Discussion). The acute inhibitory effect produced by a SSRI on DRN 5-HT neuronal activity results from increased extracellular levels of 5-HT, due to the blockade of its reuptake, and is selectively related to the stimulation of somatodendritic 5-HT_{1A} autoreceptors (Hajos et al., 1999). The degree of inhibition induced by citalopram has therefore been used as a reliable index of their sensitivity (Arborelius et al., 1999, 2004; Sanchez et al., 2003). It appears that a 3-day treatment with a 5-HT₄ receptor agonist is sufficient to desensitize 5-HT_{1A} autoreceptors in the DRN.

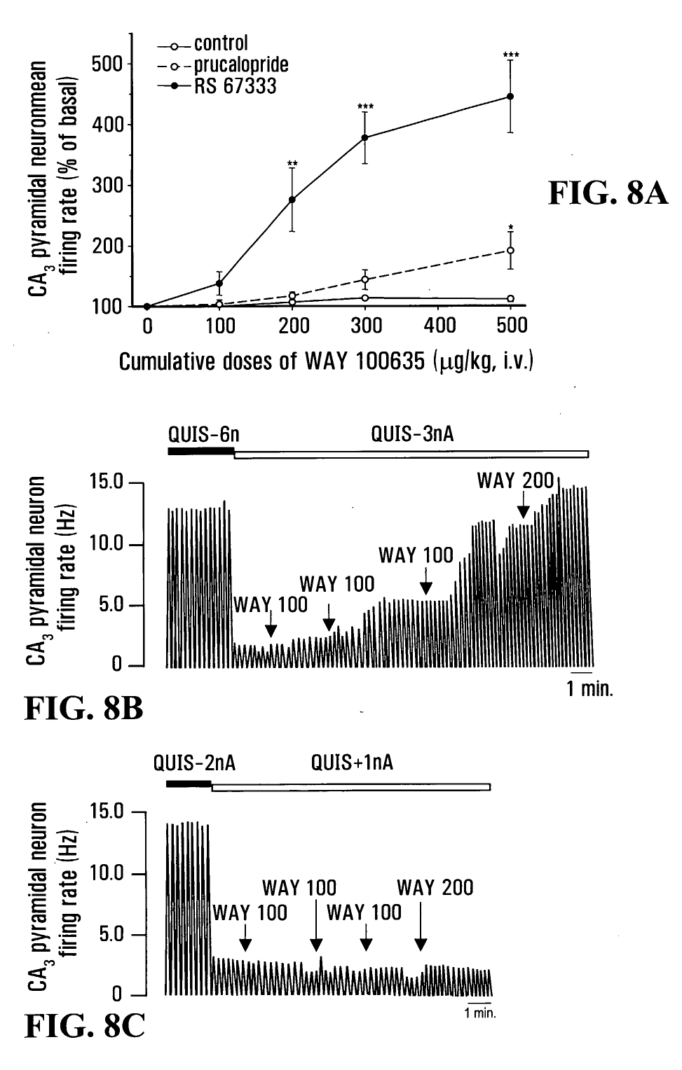

Effect of 3-day treatments with 5-HT₄ agonists on the response of hippocampal pyramidal neurons to a 5-HT_{1A} antagonist. The firing activity of dorsal hippocampus pyramidal neurons was then recorded in the CA₃ subfield, before and after systemic administration of the selective 5-HTLA antagonist WAY 100635. This protocol has proved useful to reveal an increased tonic stimulation of inhibitory postsynaptic 5-HT_{1A} receptors, a common and selective trait for all AD treatments (Haddjeri et al., 1998; Besson et al., 2000; Blier and Ward, 2003). As illustrated in FIG. 8A, cumulative (100-500 μg/kg) i.v. injections of WAY 100635 had no effect on CA₃ pyramidal neurons in vehicle-treated animals, confirming that, in normal conditions of anaesthesia, there is virtually no 5-HT_{1A} tone in this brain area (Haddjeri et al., 1998; Besson et al., 2000). However, WAY 100635 had a dose-dependent excitatory effect in rats treated with either RS 67333 (1.5 mg/kg/day) or prucalopride (2.5 mg/kg/day), another selective 5-HT₄ agonist (Briejer et al., 2001) (FIG. 8A). The effect was moderate in the prucalopride group, reaching statistical significance only after the highest cumulative i.v. dose of WAY 100635 (500 μg/kg), at 191 (±31) % of baseline. In contrast, in the RS 67333 group, all three cumulative doses of 200, 300, and 500 μg/kg strongly increased the firing activity of CA₃ pyramidal neurons to 276 (±52), 378 (±43) and 445 (±60) % of basal values, respectively (FIGS. 8A and 8B). Three days of treatment with 5-HT₄ agonists was therefore sufficient for 5-HT_{1A} receptors to exert a tonic inhibitory influence on CA₃ pyramidal neurons. The decreased basal activity of these neurons was confirmed by comparing the ejection currents of quisqualate required to perform the experiments. Indeed, as most hippocampal pyramidal neurons are not spontaneously active under chloral hydrate anaesthesia, a leak or a small ejection current (0 to −2 nA) of quisqualate (through the use of a multi-barrelled electrode and microiontophoretic “pumps”, see Methods) is necessary to activate them within their physiological firing range of 10-15 Hz (Haddjeri and Blier, 1995; Haddjeri et al., 1998). In the RS 67333-treated group, their excitability was reduced to such an extent that currents as high as −4 to −6 nA were required to obtain similar basal values (FIG. 8B). Again, the effect was less prominent in the prucalopride group, since the required currents were between −2 and −4 nA (not shown).

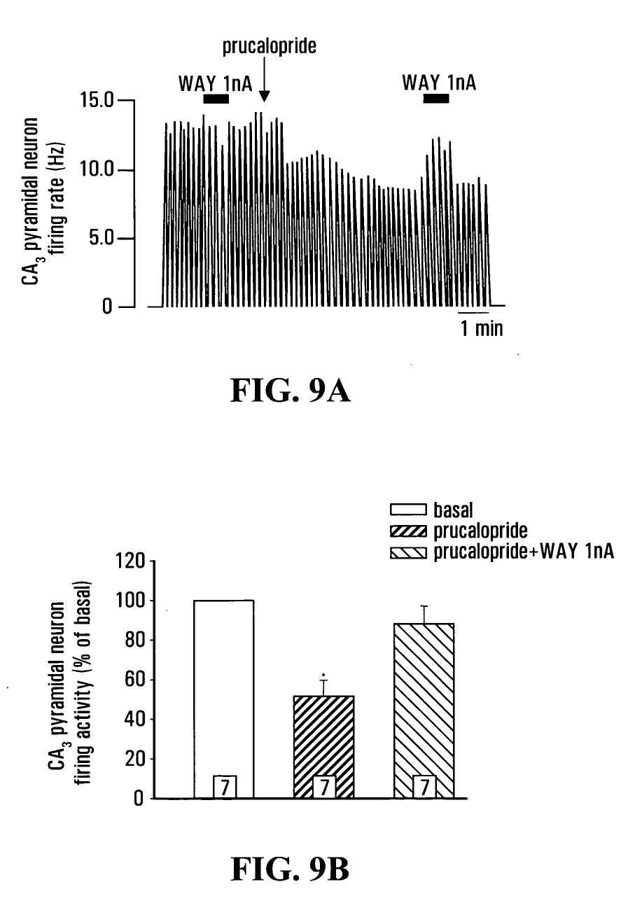

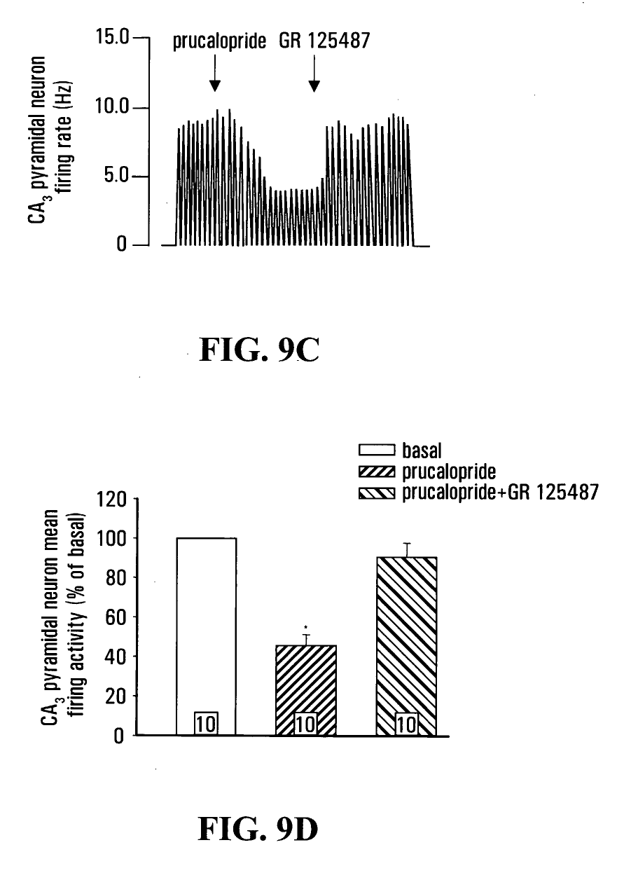

Involvement of endogenous 5-HT and hippocampal 5-HT_{1A} receptors in the effect of 5-HT₄ agonists. The most logical explanation for the above results would be that short-term treatments with 5-HT₄ agonists induced an increase of 5-HT release in the CA₃ sub-region, which in turn resulted in an enhanced stimulation of postsynaptic 5-HT_{1A} receptors located on pyramidal neurons. This interpretation is consistent with our own previous findings, showing that both acute and chronic (3-day) administration of the same compounds augment DRN 5-HT neuronal firing activity (Lucas and Debonnel, 2002; Lucas et al., 2005). It is also in keeping with observations from a single in vivo microdialysis study, where acute systemic injection of preferential 5-HT₄ agonists increased hippocampal 5-HT efflux (Ge and Barnes, 1996). To further address this possibility, two additional series of experiments were conducted. First, the role of endogenous 5-HT was assessed by using the same protocol as above, in rats co-treated with RS 67333 (or its vehicle) and the 5-HT depleter para-chlorophenylalanine (pCPA). In the vehicle group, pCPA had no effect per se on both CA₃ neuron firing activity and their response to WAY 100635 (not shown; n=4). However, as shown in FIG. 8C, pCPA totally abolished the facilitory action of WAY 100635 in rats treated with RS 67333 (n=5). The ejection currents of quisqualate needed to activate CA₃ pyramidal neurons were, in this case, similar to those used in controls (0 to −2 nA, FIG. 8C). We then tested the ability of a local application of WAY 100635, directly into the CA₃ subfield through the use of microiontophoresis, to modify the effect of a 5-HT₄ agonist. We chose not to use the previous protocol (i.e. chronic treatments), because, as shown above, the required quisqualate currents differ between the experimental groups. Even though all currents were balanced, it was not possible to exclude the possibility that the amount of quisqualate present in the vicinity of the electrode may have an influence on the quantity of WAY 100635 actually ejected, and/or on its ability to act on pyramidal neurons. For this reason, prucalopride was used acutely in naïve rats, at a dose known to induce a significant augmentation of DRN 5-HT neuronal activity (1000 μg/kg, i.v.) (Lucas and Debonnel, 2002). On the 38 pyramidal neurons recorded, 21 were unaffected by prucalopride, whereas the remaining 17 ones (45% of total) were clearly inhibited, discharging at only 46 (±5.6) % of pre-drug values (FIG. 9). This inhibition was totally reversed by the microiontophoretic application of WAY 100635 (1 nA), devoid of any effect by itself (FIGS. 9A and 9B). The involvement of 5-HT₄ receptors in the action of prucalopride was confirmed, since it was abolished by systemic administration of the selective 5-HT₄ antagonist GR 125487 (1000 μg/kg, i.v., FIGS. 9C and 9D).

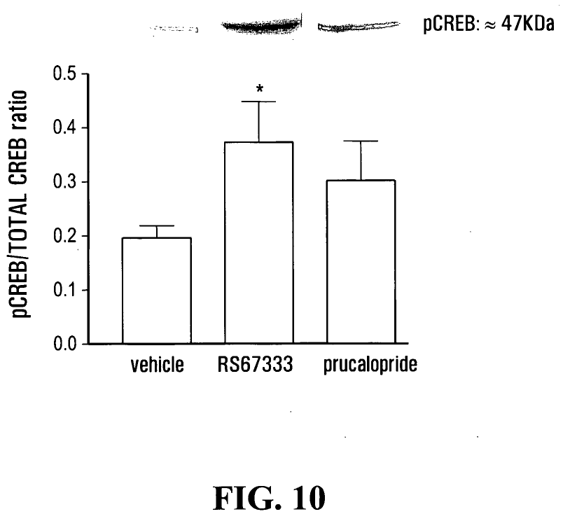

Effect of 3-day treatments with 5-HT₄ agonists on CREB phosphorylation in the hippocampus. These latter results confirmed our proposed cascade of events, with a critical role for DRN-hippocampus 5-HT transmission in mediating the observed effects. The next step was to assess whether these changes in 5-HT neurotransmission were also accompanied by modifications of the molecular dynamics taking place downstream to membrane signalling processes. Indeed, ADs known to directly enhance 5HT extracellular levels, such as SSRIs, affect the cAMP transduction pathway in hippocampal neurons, and increase activation of CREB, only after at least two weeks of chronic administration (Nibuya et al., 1996; Thome et al., 2000; Tiraboschi et al., 2004). In contrast, after a 3-day treatment with 5-HT₄ agonists, we found that the pCREB/CREB ratio in hippocampal tissue was enhanced (FIG. 10), with no changes in the total amount of CREB present in the samples (not shown). The mean (±S.E.M.) values of ratios were 0.196±0.02, 0.302±0.07 and 0.373±0.07 in the control, prucalopride and RS 67333 groups, respectively. The increase observed in RS 67333-treated animals (+90%) was statistically significant (p<0.05, one-way ANOVA), whereas the +54% effect found in the prucalopride group failed to reach significance (p=0.07).

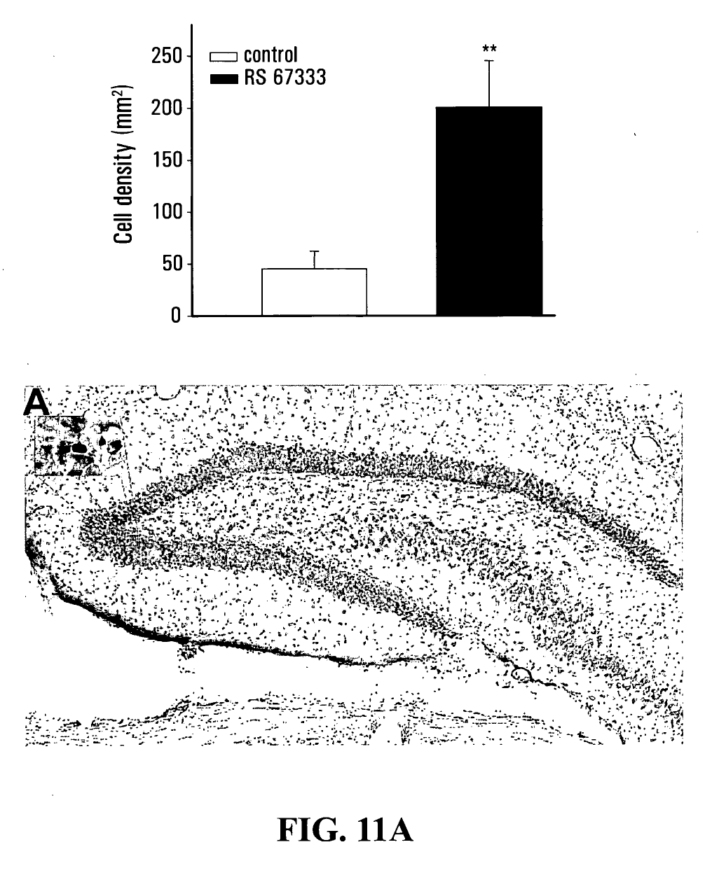

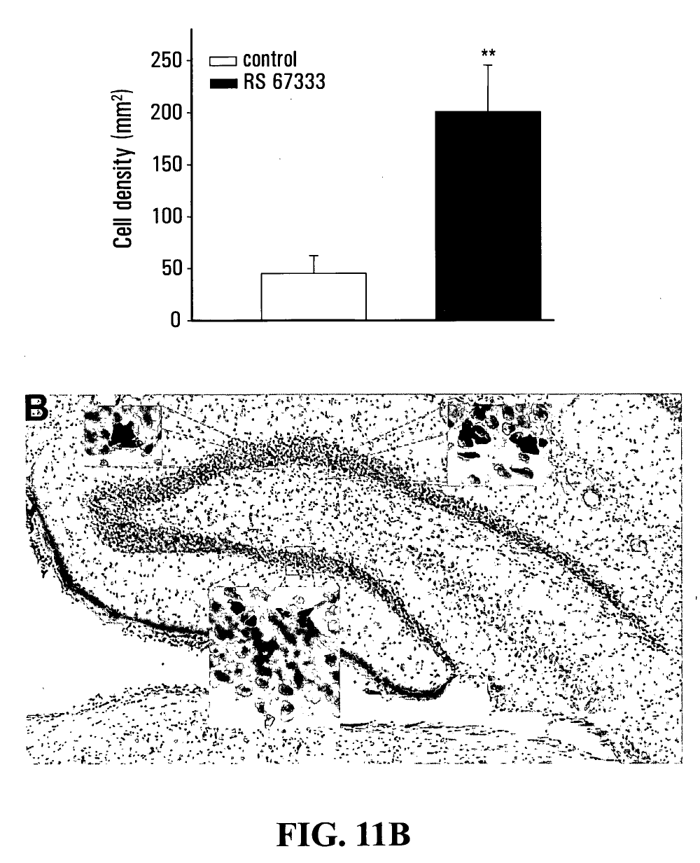

Effect of a 3-day treatment with RS 67333 on hippocampal neurogenesis. Classical ADs, and especially SSRIs, promote adult neurogenesis in the hippocampus (Malberg et al., 2000; Duman et al., 2001; Nestler et al., 2002; Castrén, 2004). Increased neurogenesis occurs predominantly in the sub-granular zone (SGZ) of the dentate gyrus (Malberg et al., 2000; Nakagawa et al., 2002a; Santarelli et al., 2003), and requires at least 2 or 3 weeks of chronic treatment (Malberg et al., 2000; Nakagawa et al., 2002a; Santarelli et al., 2003). Remarkably, a 3-day treatment with RS 67333 strongly enhanced the number of bromodeoxyuridine (BrdU)-positive cells in the SGZ (FIG. 11). The density of BrdU-positive cells (per mm²) increased from 44.9±17.2 (mean±S.E.M., n=4) in control animals up to 200.2±44.9 (mean±S.E.M., n=4) in the RS 67333 group, accounting for a dramatic increase of 346%. Detailed morphological analysis of the histological sections revealed that these cells were grouped in clusters of 6 to 10 clones following the RS 67333 treatment, suggesting an intense acute induction of mitotic activity (FIG. 11B). By contrast, there were virtually no clusters of BrdU-positive cells in the control group (FIG. 11A).

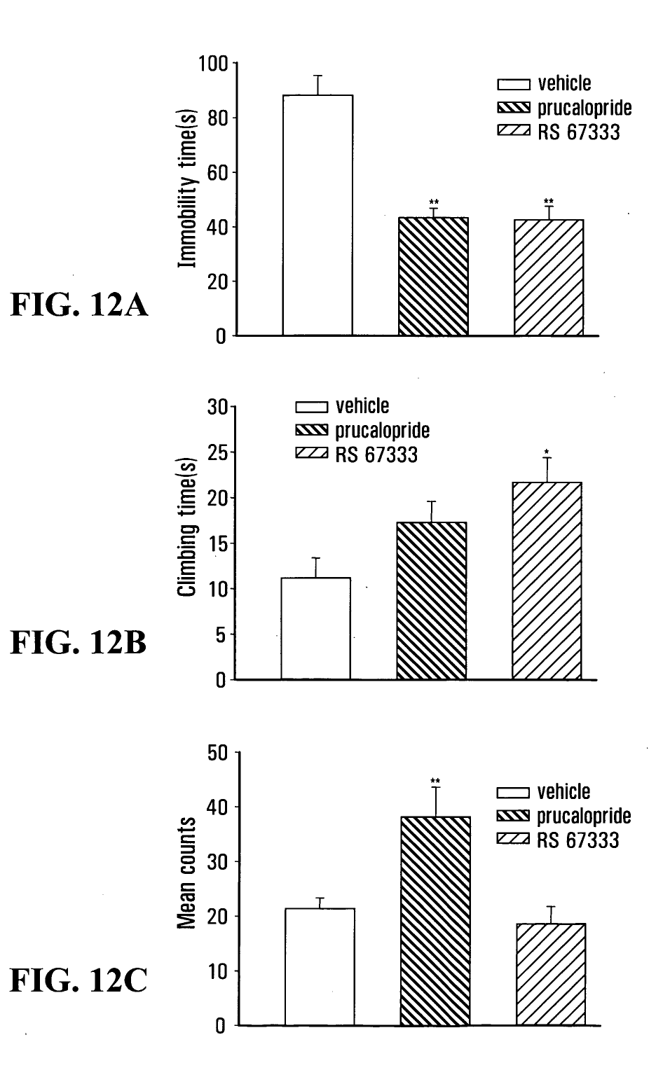

Effect of RS 67333 in the FST. Finally, the efficacy of the 5-HT₄ agonists used was also tested in the FST, which has proven a highly reliable predictor of antidepressant potential (Cryan et al., 2002). As shown in FIG. 12A, both prucalopride and RS 67333 strongly reduced the time of immobility, to a similar extent (around 50%): from 88.2±7.2 (control rats) to 43.4±3.5 and 42.7±4.9 seconds, respectively. However, RS 67333 was again more potent than prucalopride in facilitating climbing, as only its effect (+94%) reached statistical significance (FIG. 12B). The FST has to be validated by also testing the influence of the compounds used on motor behavior, because an increase in motor activity could have also accounted for the observed results (Cryan et al., 2002; Haddjeri et al., 2004). As shown in FIG. 12C, RS 67333 had no effect on rat locomotion; however, prucalopride increased significantly this parameter, by 78%. We could not therefore exclude that the behavioral effects induced by prucalopride were at least in part related to a motor component.

### Discussion

Our results indicate that electrophysiological, molecular and morphological changes that have previously been specifically linked to long-term treatment with SSRIs are already present after only 3 days when using 5-HT₄ agonists. Consistent with the facilitory action displayed by the latter, at the same dose regimen, on DRN 5-HT neuron firing rate (Lucas et al., 2005), we found that the DRN-hippocampus 5-HT transmission was deeply modified by 5-HT₄ receptor activation. The shift to the right in the ability of citalopram to inhibit 5-HT neuron firing rate (FIG. 7) clearly suggests a decreased sensitivity of somatodendritic 5-HT_{1A} autoreceptors. It could still be argued that this shift only resulted from the arithmetic sum of two opposite influences, namely an inhibition caused by citalopram, versus the facilitory effect of the 5-HT₄ agonist. However, as stated above, this effect was observed in all the neurons recorded. We have previously shown that a subpopulation, representing about one-half of DRN 5-HT neurons and discharging at a low (≦1.0 Hz) basal frequency, is not sensitive to 5-HT₄ receptor stimulation (Lucas and Debonnel, 2002). It appears therefore that the reduced sensitivity to citalopram observed in all neurons was related to a “true” desensitization of 5-HT_{1A} autoreceptors. This strongly suggests that 5-HT extracellular levels were enhanced in the DRN after a 3-day treatment with RS 67333, as already proposed to explain the desensitization induced by SSRIs (Blier and de Montigny, 1999; Artigas et al., 2002) which is known to progress gradually, being observable only after 2-3 weeks (Blier and de Montigny, 1994; El Mansari et al., 2005). The more rapid desensitization observed with RS 67333 may be due to the different characteristics of the mechanisms involved: rather than a diffuse, passive elevation of 5-HT extracellular levels consequent to the blockade of 5-HT reuptake sites, the activation of 5-HT neuronal firing rate elicited by 5-HT₄ agonists should facilitate a true, active, release of 5-HT within the DRN.

This possibility is also consistent with our conclusion that 5-HT release was enhanced in projection areas, more particularly the dorsal hippocampus, after a 3-day treatment with 5-HT₄ agonists. This enhancement was unveiled by the manifestation of an inhibitory tonus, mediated by endogenous 5-HT through the stimulation of postsynaptic 5-HT_{1A} receptors (FIG. 8). Such an effect has already been observed with classical AD treatments (such as SSRIs, tricyclics, electroconvulsive shocks), but, again, only after 2-3 weeks of sustained administration (Haddjeri et al., 1998; Besson et al., 2000). Moreover, in the case of RS 67333, the inhibitory tonus appeared to be even stronger than that induced by a 21-day treatment with SSRIs. Indeed, the selective 5-HT_{1A} antagonist WAY 100635 was able to increase CA₃ pyramidal neuron activity up to 440% of pre-drug values (FIG. 8), whereas this disinhibition has been reported to reach about 200% after 3 weeks treatment with the SSRI paroxetine (Haddjeri et al., 1998). The effect of prucalopride, on the other hand, was more modest, as a high cumulative dose of WAY 100635 was needed to unveil the existence of the 5-HT_{1A}-mediated tonus. The difference of amplitude between the effects of the two 5-HT₄ agonists is in agreement with their respective action on DRN 5-HT neuron mean firing rate: prucalopride enhanced this parameter by about 40-45%, whereas the effect of RS 67333 reached 70% (Lucas et al., 2005). It is therefore possible that the increase of 5-HT release induced by prucalopride in the hippocampus was less important than that elicited by RS 67333. It is of interest to mention that the dose of prucalopride used for chronic treatments is relatively low, in that this compound appears to be still selective for 5-HT₄ receptors at the dose of 5 mg/kg, i.p. (Durier et al., 2000; Porras et al., 2002). In contrast with these modest short-term effects, the acute injection of 1000 μg/kg i.v., prucalopride was able to inhibit the activity of CA₃ pyramidal neurons by almost 50%, but only in 45% of the cases (FIG. 9). This result remarkably parallels the findings that, in acute conditions, the same treatment strongly increase (around 100%) DRN 5-HT activity, but only in 47% of 5-HT neurons (that we dubbed “responders” in a previous report) (Lucas and Debonnel, 2002). Considering the very sparse, diffuse nature of central 5-HT innervation, it seems unlikely that “responders” project exclusively on certain CA₃ pyramidal neurons. However, the above results may be related to the fact that, overall, 5-HT₄ receptors exert a dual action on these cells. Indeed, the direct application of a 5-HT₄ agonist onto pyramidal neurons, through the use of microiontophoresis, has been shown to actually increase their firing activity (Lucas et al., 2005). On the other hand, as discussed above, the stimulation of other 5-HT₄ receptors, notably in the medial prefrontal cortex (Lucas et al., 2005), induces the 5-HT_{1A}-mediated inhibitory effect through an indirect mechanism involving 5-HT neurons. The net influence resulting from the systemic injection of a 5-HT₄ agonist will therefore depend on this functional balance between positive and negative actions of central 5-HT₄ receptors. None of the 38 CA₃ neurons recorded was excited by the acute administration of prucalopride, implying that, even when a pyramidal neuron is innervated by a majority of “non-responders”, the indirect, inhibitory effect is strong enough to cancel the positive action of hippocampal 5-HT₄ receptors. Moreover, after a chronic 5-HT₄ agonist administration, all of the pyramidal neurons encountered were inhibited. This indicates that the inhibitory influence amplifies progressively in the CA₃ sub-field. This predominance of the indirect effect of 5-HT₄ receptors further illustrates the major role, unsuspected until recently, that the 5-HT₄-mediated control of 5-HT neurons plays in the regulation of the central 5-HT system. It is also of crucial importance in a therapeutic perspective, an increased 5-HT_{1A}-mediated inhibition of hippocampal cells being considered a crucial step to obtain the beneficial effects of ADs such as SSRIs (Haddjeri et al., 1998; Santarelli et al., 2003; Castrén, 2004).

In line with this statement, we found that both prucalopride and RS 67333 were able, after only 3 days of chronic administration, to enhance the pCREB/CREB ratio in hippocampal tissue. Such an effect has also been reported to constitute a specific marker of AD action in rat brain (Nibuya et al., 1996; Thome et al., 2000; Nakagawa et al., 2002a; Tiraboschi et al., 2004), but, again, has never been observed before 1.5-2 weeks of treatment (Nibuya et al., 1996). Here, the effect of RS 67333 was stronger than that of prucalopride, paralleling electrophysiological results, but more importantly, it was also of higher amplitude (+94%, FIG. 10) than the previously reported increase induced by a SSRI (+20-30%) (Tiraboschi et al., 2004). Several studies have shown that the pro-neurogenetic action of SSRIs is directly related to their ability to phosphorylate CREB (Nakagawa et al., 2002a,b), and that an increased neurogenesis is crucial for the AD potential of these compounds (Santarelli et al., 2003). For this reason, we tested whether a 3-day treatment with RS 67333, the most active of the 5-HT₄ agonists used in electrophysiological and molecular experiments, was also able to facilitate neurogenesis in the hippocampus. The results shown in FIG. 11 clearly confirm the ability of such a treatment to enhance cell division and proliferation in the SGZ. Moreover, the effect of 3-day RS 67333 (+346%) was much stronger than that reported for a 14-day treatment with the SSRI fluoxetine (+35%) (Malberg et al., 2000), in conformity with what we found on PCREB. To our knowledge, this is the first time that a compound known to facilitate 5-HT transmission is shown to activate adult hippocampal neurogenesis so rapidly. So far, the quickest effect reported was 11 days for fluoxetine, which was inactive at 5 days (Santarelli et al., 2003); with other ADs, neurogenesis occurred after 2-3 weeks only (Malberg et al., 2000; Castrén, 2004). On the basis of these studies, it has been proposed that the delay of action of AD could be not directly related to 5-HT transmission, but would be due to the “inertia” of the transductional and molecular changes that are required for neuronal proliferation and plasticity to occur (Manji and Duman, 2001; Manji and Zarate, 2002; Nestler et al., 2002, Castrén, 2004). According to this hypothesis, it has been suggested that the research for more rapid AD should focus on compounds able to boost these molecular mechanisms, rather than on new ways to act on 5-HT (and more generally, aminergic) neurotransmission (Manji and Duman, 2001; Manji and Zarate, 2002; Nestler et al., 2002). However, the present study demonstrates that both CREB phosphorylation and hipoccampal neurogenesis can be achieved as rapidly as after 3 days of treatment, concomitant with an increase of 5-HT neuron firing rate. This conclusion emphasizes the central role of the 5-HT system in AD research, in agreement with alternative proposals (Blier, 2001; Artigas et al., 2002). That 5-HT₄ agonists have properties common to other ADs was further confirmed by the results obtained in the FST. Again, the reduction of immobility time we found in this test for both RS 67333 and prucalopride (−50%) was of much greater amplitude than what is commonly observed with SSRIs (around −25%, not significant in all the studies) (Cryan et al., 2002; Haddjeri et al., 2004). The effect induced by prucalopride, however, was also paralleled by an increase of motor activity. It is possible that the low affinity displayed by this compound for dopamine-D₄ receptors (Briejer et al., 2001) may account for this result, since even a weak stimulation of D₄ receptors has been reported to facilitate locomotion (Nayak and Cassaday, 2003). Whatever its exact causes, this non-specific action of prucalopride prompted us to not use this drug at a higher dose than 2.5 mg/kg, even if it may not have been maximal with respect to 5-HT₄ receptor stimulation (see above). It constitutes also one of the reasons for the present study to be more focused on the use RS 67333, which, again in the FST, appeared to be more efficient and promising.

### Example 7

### Assessment of the Effect of the 5-HT₄ Receptor Agonists, Alone or in Combination with a SSRI on 5-HT₁ₐ Tonus in the Hippocampus

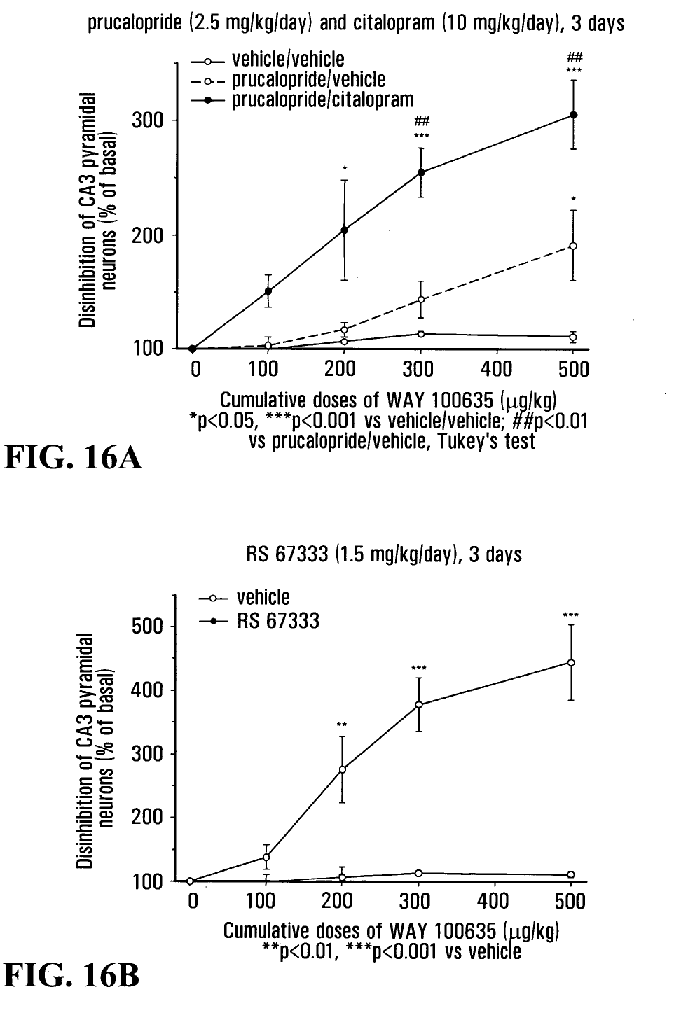

The firing activity of dorsal hippocampus pyramidal neurons was recorded in the CA₃ subfield, before and after systemic administration of the selective 5-HT_{1A} antagonist WAY 100635 (see FIGS. 16A and 16B). This protocol has proved very reliable and useful to reveal an increased tonic stimulation of inhibitory postsynaptic 5-HT_{1A} receptors in this brain area, which constitutes a common and selective trait for all AD treatments. Thus, in conditions of anaesthesia, there is virtually no 5-HT_{1A} tone in the hippocampus of control animals; consequently, cumulative doses of WAY 100635 remained without any effect on pyramidal neuron activity. Conversely, the fact that WAY 100635 had a dose-dependent facilitory effect in rats treated for 3 days with either prucalopride alone, prucalopride and citalopram, or RS 67333 alone, indicates that these treatments induced the manifestation of a 5-HT_{1A}-mediated inhibitory tonus in the hippocampus. The more the effect of WAY 100635 has a high amplitude, the stronger is the inhibitory tonus. Thus, our results reveal that prucalopride alone elicited a modest inhibitory tonus, which was strongly potentiated when co-administered with citalopram (FIG. 16A). The administration of RS 67333 alone (FIG. 16B) was sufficient to induce a strong tonic inhibition of hippocampal pyramidal neurons. Beyond the differences in amplitude observed between these 3 experimental groups, the most important finding remains that all of them were able to induce the manifestation of the 5-HT_{1A}-mediated inhibitory tonus after only 3 days of treatment. Several studies have shown that classical antidepressants, including SSRIs, are unable to induce such an effect before 2, or even 3 weeks.

Overall, the results presented here show a clear potential for 5-HT₄ agonists as putative antidepressants with a rapid onset of action. According to the different experimental models studied, they may act 5 to 7 times more rapidly than classical ADs, and possibly with a greater efficacy. Presently, RS 67333 and prucalopride are virtually the only selective 5-HT₄ agonists able to cross the blood-brain barrier that are available.

The following references are cited throughout the specification. All documents mentioned herein are incorporated herein by reference.

### REFERENCES

Aghajanian, G. K. (1978). Essays in Neurochemistry and Neuropharmacology, M. B. H. Youdim, W. Lovenbery, D. F. Sharman, and J. R. Lagnado, eds. (New-York, N.Y.: Wiley), pp. 1-32.
Arborelius, L., Wallsten, C., Ahlenius, S., and Svensson, T. H. (1999). Eur. J. Pharmacol. 382, 133-138.
Arborelius, L., Hawks, B. W., Owens, M. J., Plotsky, P. M., and Nemeroff, C. B. (2004). Psychopharmacology 176, 248-255.
Besson, A., Haddjeri, N., Blier, P., and de Montigny, C. (2000). Eur. Neuropsychopharmacol. 10, 177-188.
Blier, P. (2001). J. Clin. Psychiatry 62(Suppl. 4), 7-11.
Blier, P., and de Montigny, C. (1994). Trends Pharmacol. Sci. 15, 220-226.
Blier, P., and de Montigny, C. (1999). Neuropsychopharmacology 21(Suppl.), 91S-98S.
Blier, P., and Ward, N. M. (2003). Biol. Psychiatry 53, 193-203.
Briejer, M. R., Bosmans, J. P., Van Daele, P., Jurzak, M., Heylen, L., Leysen, J. E., Prins, N. H., and Schuurkes, J. A. (2001). Eur. J. Pharmacol. 423, 71-83.
Castrén, E. (2004). Curr. Opin. Pharmacol. 4, 58-64.
Cervo L, Samanin R (1987). European Journal of Pharmacology. 144(2):223-229.
Cervo L, Samanin R (1991). Psychopharmacology (Berl). 103(4):524-528.
Cryan, J. F., Markou, A., and Lucki, I. (2002). Trends Pharmacol. Sci. 23, 238-245.
Descarries, L., Watkins, K. C., Garcia, S. & Beaudet, A. (1982) J. Comp. Neurol. 207, 239-254.
Detke M J, Wieland S, Lucki I (1995). Psychopharmacology (Berl). 119(1):47-54.
Duman, R. S., Nakagawa, S., and Malberg, J. (2001). Neuropsychopharmacology 25, 836-844.
Durier, S., Dahmoune, Y., Ricard, C., Jacquot, C., Langlois, M., Bourin, M., and Gardier, A. M. (2000). Soc. Neurosci. Abstr. 26, 624.10.
Eglen, R. M., Bonhaus, D. W., Johnson, L. G., Leung, E., and Clark, R. D. (1995). Br. J. Pharmacol. 115, 1387-1392.
El Mansari, M., Sanchez, C., Chouvet, G., Renaud, B., and Haddjeri, N (2005). Neuropsychopharmacology 30, 1269-1277.
Fabre, V. and Hamon, M. (2003) Encéphale, 29, 259-265.
Gale, J. D., Grossman, C. J., Darton, J., Bunce, K. T., Whitehead, J. W. F., Knight, J., Parkhouse, T. J., Oxford, A. W. & Humphrey, P. P. A. (1994) Br. J. Pharmacol. 113(Suppl.), 120P.
Ge, J., and Barnes, N. M. (1996). Br. J. Pharmacol. 117, 1475-1480.
Haddjeri, N., and Blier, P. (1995). Synapse 20, 54-67.
Haddjeri, N., Blier, P. & de Montigny, C. (1996) J. Pharmacol. Exp. Ther. 277, 861-871.
Haddjeri, N., Blier, P., and de Montigny, C. (1998). J. Neurosci. 18, 10150-10156.
Haddjeri, N., Faure, C., Lucas, G., Mnie-Filali, O., Chouvet, G., Astier, B., Renaud, B., Blier, P., and Debonnel, G. (2004). Int. J. Neuropsychopharmacol. 7, 391-399.
Jacobs, B. L. & Formal, C. A. (1999). Neuropsychopharmacology 21(Suppl.), 9S-15S.
Lamirault, L. & Simon, H. (2001) Neuropharmacology 41, 844-853.
Lucas, G., and Debonnel, G. (2002). Eur. J. Neurosci. 16, 817-822.
Lucas, G., Compan, V., Charnay, Y., Neve, R. L., Nestler, E. J., Bockaert, J., Barrot, M., and Debonnel, G. (2005). Biol. Psychiatry 15, 918-925.
Lucki I, Singh A, Kreiss D S (1994). Neuroscience and Behavioral Reviews. 18(1):85-95.
Lucki I (1997). Behavioral Pharmacology 8(6-7):523-532.
Luscombe G P, Martin K F, Hutchins L J, Gosden J, Heal D J (1993). British Journal of Pharmacology 108(3):669-677.
Malberg, J. E., Eisch, A. J, Nestler, E. J., and Duman, R. S. (2000). J. Neurosci. 20, 9104-9110.
Manji, H. K., and Duman, R. S. (2001). Psychopharmacol. Bull. 35, 5-49.
Manji, H. K., and Zarate, C. A. (2002). Mol. Psychiatry. 7(Suppl. 1), S1-S7.
Mongeau, R., Blier, P., and de Montigny, C (1997). Brain Res Brain Res. Rev. 23, 145-195.
Nakagawa, S., Kim, J.-E., Lee, R., Malberg, J. E., Chen, J., Steffen, C., Zhang, Y.-J., Nestler, E. J., and Duman, R. S. (2002a). J. Neurosci. 22, 3673-3682.
Nakagawa, S., Kim, J.-E., Lee, R., Chen, J., Fujioka, T., Malberg, J., Tsuji, S., and Duman, R. S. (2002b). J. Neurosci. 22, 9868-9876.
Nayak, S., and Cassaday, H. J. (2003). Prog. Neuropsychopharmacol. Biol. Psychiatry 27, 441-449.
Nestler, E. J., Barrot, M., DiLeone, R. J., Eisch, A. J., Gold, S. J., and Monteggia, L. M. (2002). Neuron 34, 13-25.
Nibuya, M., Nestler, E. J., and Duman, R. S. (1996). J. Neurosci. 16, 2365-2372.
Paxinos, G., and Watson, C. (1986). The rat brain in stereotaxic coordinates, 2^{nd} edition (New York, N.Y.: Academic).
Pifieyro, G., Azzi, M., De Lean, A. Schiller, P., and Bouvier, M. (2001). Mol. Pharmacol. 60, 816-827.
Porras, G., Di Matteo, V., De Deurwaerdere, P., Esposito, E., and Spampinato, U. (2002). Neuropharmacology 43, 1099-1109.
Porsolt, R. D., Le Pichon, M., and Jalfre, M. (1977). Nature 266, 730-732.
Sadikot, A. F., and Sasseville, R. (1997). J. Comp. Neurol. 389, 193-211.
Sanchez, C., Bergqvist, P. B. F., Brennum, L. T., Gupta, S., Hogg, S., Larsen, A., and Wiborg, O. (2003). Psychopharmacology 167, 353-362.
Santarelli, L., Saxe, M., Gross, C., Surget, A., Battaglia, F., Dulawa, S., Weisstaub, N., Lee, J., Duman, R., Arancio, O., Belzung, C., and Hen, R. (2003). Science 301, 805-809.
Soriano, E., and Del R10, J. A. (1991). J. Histochem. Cytochem. 39, 255-263.
Tatarczynska E, Klodzinska A, Chojnacka-Wojcik E (2002) Pol J Pharmacol. 54: 615-623.
Thome, J., Sakai, N., Shin, K.-H., Steffen, C., Zhang, Y.-J., Impey, S., Storm, D., and Duman, R. S. (2000). J. Neurosci. 20, 4030-4036.
Tiraboschi, E., Tardito, D., Kasahara, J., Moraschi, S., Pruneri, P., Gennarelli, M., Racagni, G., and Popoli, M. (2004). Neuropsychopharmacology 29, 1831-1840.
Wieland S, Lucki I (1990). Psychopharmacology (Berl). 101(4):497-504.

1. A combination comprising a 5-HT₄ agonist, or a pharmaceutically acceptable salt thereof, and a SSRI or a pharmaceutically acceptable salt thereof.

2. The combination according to claim 1, wherein the 5-HT₄ agonist is selected from the group consisting of prucalopride, RS 67333 (1-(4-amino-5-chloro-2-methoxyphenyl)-3-(1-n-burtl-4-piperidinyl)-1-propanone), RS 67506 (1-(4-amino-5-chloro-2-methoxyphenyl)-3-[1-[2-[(methylsulfonyl)amino]ethyl]-4-piperidinyl]-1-propanone), cisapride, renzapride, norcisapride, mosapride, zacopride, tegaserod, SB 205149, SC 53116, BIMU 1 and BIMU 8.

3. The combination according to claim 1, wherein the SSRI is selected from the group consisting of citalopram, escitalopram, fluoxetine, fluvoxamine, sertraline, paroxetine, zimeldine, norzimeldine, clomipramine, alaproclate, venlafaxine, cericlamine, duloxetine, milnacipran, nefazodone, OPC 14503, and cyanodothiepin.

4. The combination according to claim 1, comprising prucalopride and citalopram.

5. The combination according to claim 1, comprising RS 67333 and paroxetine.

6. The combination according to claim 1, comprising RS 67333 and fluvoxamine.

7. The combination according to claim 1, comprising RS 67333 and fluoxetine.

8. A composition comprising an effective amount of the combination as defined in claim 1, and one or more pharmaceutically acceptable carriers.

9. The composition according to claim 8, wherein the SSRI is selected from the group consisting of citalopram, escitalopram, fluoxetine, fluvoxamine, sertraline, paroxetine, zimeldine, norzimeldine, clomipramine, alaproclate, venlafaxine, cericlamine, duloxetine, milnacipran, nefazodone, OPC 14503, and cyanodothiepin.

10. The composition according to claim 8, wherein the 5-HT₄ agonist is selected from the group consisting of prucalopride, RS 67333 (1-(4-amino-5-chloro-2-methoxyphenyl)-3-(1-n-burtl-4-piperidinyl)-1-propanone), RS 67506 (1-(4-amino-5-chloro-2-methoxyphenyl)-3-[1-[2-[(methylsulfonyl)amino]ethyl]-4-piperidinyl]-1-propanone), cisapride, renzapride, norcisapride, mosapride, zacopride, tegaserod, SB 205149, SC 53116, BIMU 1 and BIMU 8.

11. The composition according to claim 8, comprising prucalopride and citalopram.

12. The composition according to claim 8, comprising RS 67333 and paroxetine.

13. The composition according to claim 8, comprising RS 67333 and fluvoxamine.

14. The composition according to claim 8, comprising RS 67333 and fluoxetine.

15. A package containing separated dosage units, of which at least one dosage unit comprises 5-HT₄ agonist and at least one other dosage unit comprises an SSRI.

16. The package according to claim 15, wherein the SSRI is selected from the group consisting of citalopram, escitalopram, fluoxetine, fluvoxamine, sertraline, paroxetine, zimeldine, norzimeldine, clomipramine, alaproclate, venlafaxine, cericlamine, duloxetine, milnacipran, nefazodone, OPC 14503, and cyanodothiepin.

17. The package according to claim 15, wherein the 5-HT₄ agonist is selected from the group consisting of prucalopride, RS 67333 (1-(4-amino-5-chloro-2-methoxyphenyl)-3-(1-n-burtl-4-piperidinyl)-1-propanone), RS 67506 (1-(4-amino-5-chloro-2-methoxyphenyl)-3-[1-[2-[(methylsulfonyl)amino]ethyl]-4-piperidinyl]-1-propanone), cisapride, renzapride, norcisapride, mosapride, zacopride, tegaserod, SB 205149, SC 53116, BIMU 1 and BIMU 8.

18. The package according to claim 15, wherein the SSRI is citalopram and the 5-HT₄ agonist is prucalopride.

19. The package according to claim 15, wherein the SSRI is RS 67333 and the 5-HT₄ agonist is paroxetine.

20. The package according to claim 15, wherein the SSRI is RS 67333 and the 5-HT₄ agonist is fluvoxamine.

21. The package according to claim 15, wherein the SSRI is RS 67333 and the 5-HT₄ agonist is fluoxetine.

22-43. (canceled)

44. A method for augmenting and/or providing faster onset of the therapeutic effect of a SSRI comprising administering to a subject to be treated with or undergoing treatment with the SSRI a therapeutically effective amount of a 5-HT₄ agonist.

45. A method for augmenting and/or providing faster onset of the therapeutic effect of a SSRI comprising administering to a subject a therapeutically effective amount of the combination as defined in claim 1.

46. A method of treating depression, anxiety or other affective disorder responsive to a SSRI, or any other compound that causes an elevation in the level of extracellular serotonin, comprising administering to a subject in need thereof: (a) a therapeutically effective amount of a SSRI alone or with any other compound that causes an elevation in the level of extracellular serotonin, to treat depression, anxiety, obsessive compulsive disorder (OCD) or other disease or pharmaceutically acceptable salt thereof, to augment and/or provide faster onset of the therapeutic effect of the SSRI, or any other compound, that causes an elevation in the level of extracellular serotonin.

47. The method according to claim 46, wherein the SSRI is selected from the group consisting of citalopram, escitalopram, fluoxetine, fluvoxamine, sertraline, paroxetine, zimeldine, norzimeldine, clomipramine, alaproclate, venlafaxine, cericlamine, duloxetine, milnacipran, nefazodone, OPC 14503, and cyanodothiepin.

48. The method according to claim 46, wherein the 5-HT₄ agonist is selected from the group consisting of prucalopride, RS 67333 (1-(4-amino-5-chloro-2-methoxyphenyl)-3-(1-n-burtl-4-piperidinyl)-1-propanone), RS 67506 (1-(4-amino-5-chloro-2-methoxyphenyl)-3-[1-[2-[(methylsulfonyl)amino]ethyl]-4-piperidinyl]-1-propanone), cisapride, renzapride, norcisapride, mosapride, zacopride, tegaserod, SB 205149, SC 53116, BIMU 1 and BIMU 8.

49. The method according to claim 46, wherein the 5-HT₄ agonist is prucalopride and the SSRI is citalopram.

50. The method according to claim 46 wherein the 5-HT₄ agonist is RS 67333 and the SSRI is paroxetine.

51. The method according to claim 46 wherein the 5-HT₄ agonist is RS 67333 and the SSRI is fluvoxamine.

52. The method according to claim 46 wherein the 5-HT₄ agonist is RS 67333 and the SSRI is fluoxetine.

53. The method according to claim 46 wherein the 5-HT₄ agonist and the SSRI are administered simultaneously.

54. The method according to claim 46 wherein the 5-HT₄ agonist and the SSRI are administered sequentially.

55. The method according to claim 46 wherein the 5-HT₄ agonist and the SSRI are administered in the same unit dosage form.

56. The method according to claim 46 wherein the 5-HT₄ agonist and the SSRI are administered in two discrete unit dosage forms.

57-62. (canceled)

63. A method of treating depression, anxiety or obsessive compulsive disorder (OCD), comprising administering to a subject in need thereof a therapeutically effective amount of a 5-HT₄ agonist, or pharmaceutically acceptable salt thereof.

64. The method according to claim 63, wherein the 5-HT₄ agonist is selected from the group consisting of prucalopride, RS 67333 (1-(4-amino-5-chloro-2-methoxyphenyl)-3-(1-n-burtl-4-piperidinyl)-1-propanone), RS 67506 (1-(4-amino-5-chloro-2-methoxyphenyl)-3-[1-[2-[(methylsulfonyl)amino]ethyl]-4-piperidinyl]-1-propanone), cisapride, renzapride, norcisapride, mosapride, zacopride, tegaserod, SB 205149, SC 53116, BIMU 1 and BIMU 8.

65. The method according to claim 63 wherein the 5-HT₄ agonist is RS 67333.
